(19) United States
(12) Patent Application Publication
(10) Pub. No.: US 2015/0253330 A1
(43) Pub. Date: Sep. 10, 2015

(54) PRE-SELECTION OF SUBJECTS FOR THERAPEUTIC TREATMENT WITH AN HSP90 INHIBITOR BASED ON HYPOXIC STATUS

(71) Applicants: Vojo Vukovic, Winchester, MA (US); Ilker Yalcin, Boston, MA (US)

(72) Inventors: Vojo Vukovic, Winchester, MA (US); Ilker Yalcin, Boston, MA (US)

(73) Assignee: Synta Pharmaceuticals Corp., Lexington, MA (US)

(21) Appl. No.: 14/515,088

(22) Filed: Oct. 15, 2014

Related U.S. Application Data
(63) Continuation of application No. PCT/US2013/041107, filed on May 15, 2013.
(60) Provisional application No. 61/815,082, filed on Apr. 23, 2013.
(60) Provisional application No. 61/647,845, filed on May 16, 2012.

(51) Int. Cl. G01N 33/574; C12Q 1/68

(52) U.S. Cl. CPC G01N 33/57484; C12Q 1/6886; G01N 2800/52; G01N 2333/904; G01N 2333/475; G01N 2333/90203; G01N 2333/988; C12Q 2600/158; C12Q 2600/106; G01N 2333/71; G01N 2800/7038

(57) ABSTRACT

The present invention provides methods for the pre-selection of a subject for therapeutic treatment with an Hsp90 inhibitor based on modulated levels of hypoxia in cancerous cells in the subject. In one embodiment, the invention provides methods for the pre-selection of a subject for therapeutic treatment with an Hsp90 inhibitor based on modulated levels of lactate dehydrogenase (LDH) in a cell, e.g., a cancerous cell. The invention also provides methods for treating cancer in a subject by administering an effective amount of an Hsp90 inhibitor to the subject, wherein the subject has been selected based on a modulated level of hypoxia. The invention further provides kits to practice the methods of the invention.

### RELATED APPLICATIONS

This application claims the benefit of priority to U.S. Provisional Patent Application Nos. 61/647,845, filed on May 16, 2012; and 61/815,082, filed on Apr. 23, 2013. The contents of each of the above applications are incorporated herein by reference in their entireties.

### BACKGROUND OF THE INVENTION

As tumors grow, they begin to exceed their supply of oxygen. Hypoxia occurs when the growth of the tumor exceeds new blood vessel formation, and the tumor must undergo genetic and adaptive changes to allow it to survive and proliferate in a less well-oxygenated environment. In such a hypoxic microenvironment, tumors exhibit a greater dependency on certain signaling pathways, referred to as oxygen-sensitive pathways, to facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002).

A number of oxygen-sensitive pathways have been shown to be regulated by hypoxia, including hypoxia-inducible factor (HIF) pathways, vascular endothelial growth factor (VEGF) pathways, and mammalian target of rapamycin (mTOR) pathways. See e.g., Melillo, Cancer Metastasis Rev 26: 341-352, 2007. Hypoxia has also been shown to up-regulate epidermal growth factor receptor (EGFR) expression in tumors (Franovic et al., PNAS 104:13092-13097, 2007), which then leads to phosphorylation of tyrosine residues in the kinase domain of the receptor and activation of the Ras/Maf/MAPK or PI3K/Akt/mTOR pathways. Activation of these oxygen-sensitive pathways results in the nuclear activation of genes related to angiogenesis, cell proliferation, growth, metastasis, and adhesion (Langer and Soria, Clin. Lung Cancer, 11(2) 82-90, 2010).

Therapeutic agents targeting these oxygen-sensitive pathways are invaluable for the treatment of diseases such as cancer. However, patient response to currently available therapeutic agents is not always predictable. Indeed, although research has provided physicians with ever more options for therapeutics for the treatment of cancer, the ability to match a therapeutic agent to a specific patient based not just on the site of the tumor, but the characteristic of the tumor, is lacking. Accordingly, a need exists for the accurate prediction of patient response to currently available therapeutic agents.

### SUMMARY OF THE INVENTION

High levels of hypoxia in tumors, e.g., cells within a tumor, in a subject can be used to predict whether a patient will respond to treatment with an Hsp90 inhibitor, as disclosed herein. Specifically, the present invention provides methods for the pre-selection of a subject for therapeutic treatment with an agent based on high levels of hypoxia in cancerous cells in the subject. In one embodiment, the invention provides methods for the pre-selection of a subject for therapeutic treatment with a selected agent based on high levels of lactate dehydrogenase (LDH) in a cell, e.g., a cancerous cell. The invention also provides methods for treating cancer in a subject by administering an effective amount of an Hsp90 inhibitor to the subject, wherein the subject has been selected based on a high level of hypoxia. The invention further provides kits to practice the methods of the invention.

The invention also provides compositions for use in methods of treating a subject having cancer, the composition comprising an Hsp90 inhibitor, wherein the cancer comprises a tumor with a high level of hypoxia.

The invention also provides methods and use of a level of hypoxia in a tumor for identifying a subject for treatment with an Hsp90 inhibitor by determining the level of hypoxia in a tumor from the subject, wherein a high level of hypoxia in the sample indicates the subject is likely to respond to therapy with an Hsp90 inhibitor.

The invention also provides methods and uses of an Hsp90 inhibitor for preparation of a medicament for treating a subject having cancer, wherein the subject has a tumor with a high level of hypoxia.

The invention also provides business methods for decreasing healthcare costs by determining the level of hypoxia in a biological sample from a tumor obtained from a subject; storing the information on a computer processor; determining if the subject would likely benefit from treatment with an Hsp90 inhibitor based on the level of hypoxia; and treating the subject only if the subject will likely benefit from treatment, thereby decreasing healthcare costs.

The invention provides methods for identifying a subject for treatment with an Hsp90 inhibitor, comprising obtaining a subject sample from the subject, determining the level of hypoxia in a tumor from the subject in vitro, wherein a high level of hypoxia in the sample indicates the subject is likely to respond to therapy with an Hsp90 inhibitor.

In certain embodiments, a subject having a low level of hypoxia in the tumor is not likely to respond to therapy with an Hsp90 inhibitor.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is in the subject. In certain embodiments, the tumor tissue is removed from the subject.

In certain embodiments, the level of hypoxia is determined by detecting the activity level or expression level of one or more hypoxia-modulated polypeptides. In certain embodiments, the activity level or expression level of the one or more hypoxia-modulated polypeptides are up regulated in the sample. The level of hypoxia can be determined by any method known in the art including, but not limited to, detecting the activity level or expression level of one or more hypoxia-modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more of LDH5, LDH4, LDH3, LDH2, LDH1, LDHA and LDHB; or any combination thereof including total LDH. In certain embodiments, the isoform of HIF comprises one or more of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH that may be total LDH, LDH5, LDH4, LDH5 plus LDH4, LDH5 plus LDH4 plus LDH3, or LDHA, wherein the activity level or expression level is 0.8 ULN or more. In certain embodiments, detection of a high level of activity or expression of at least one LDH isoform or subunit comprises detection of an LDH activity or expression level of an LDH that may be total LDH, LDH5, LDH4, LDH5 plus LDH4, LDH5 plus LDH4 plus LDH3, or LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia-modulated polypeptides. In certain embodiments, a high level of hypoxia comprises a ratio or a normalized ratio of 1.0 or more of the ULN, wherein the ratio or normalized ratio may be LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, or LDH3 to total LDH.

In certain embodiments, the subject was previously treated with another chemotherapeutic agent.

In certain embodiments, the HSP90 inhibitor may be one or more of a

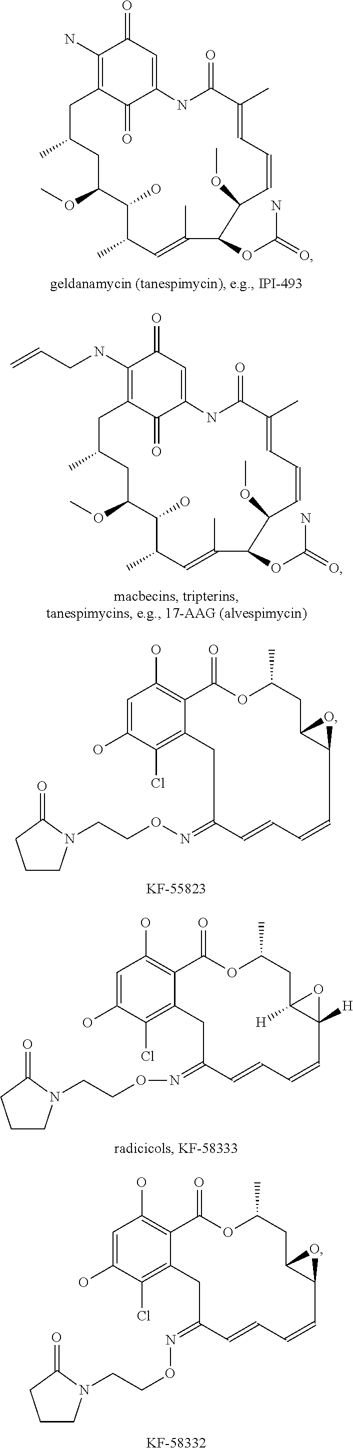 [SMILES: CO[C@H]1/C=C\C=C(/C)C(=O)NC2=CC(=O)C(N)=C(C[C@@H](C)C[C@H](OC)[C@H](O)[C@@H](C)/C=C(\C)[C@@H]1OC(N)=O)C2=O ; C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O ; C[C@@H]1C[C@@H]2O[C@H]2\C=C/C=C/C(=N/OCCN2CCCC2=O)Cc2c(Cl)c(O)cc(O)c2C(=O)O1 ; C[C@@H]1C[C@H]2O[C@@H]2\C=C/C=C/C(=N/OCCN2CCCC2=O)Cc2c(Cl)c(O)cc(O)c2C(=O)O1 ; C[C@@H]1C[C@@H]2O[C@H]2\C=C/C=C/C(=N\OCCN2CCCC2=O)Cc2c(Cl)c(O)cc(O)c2C(=O)O1]

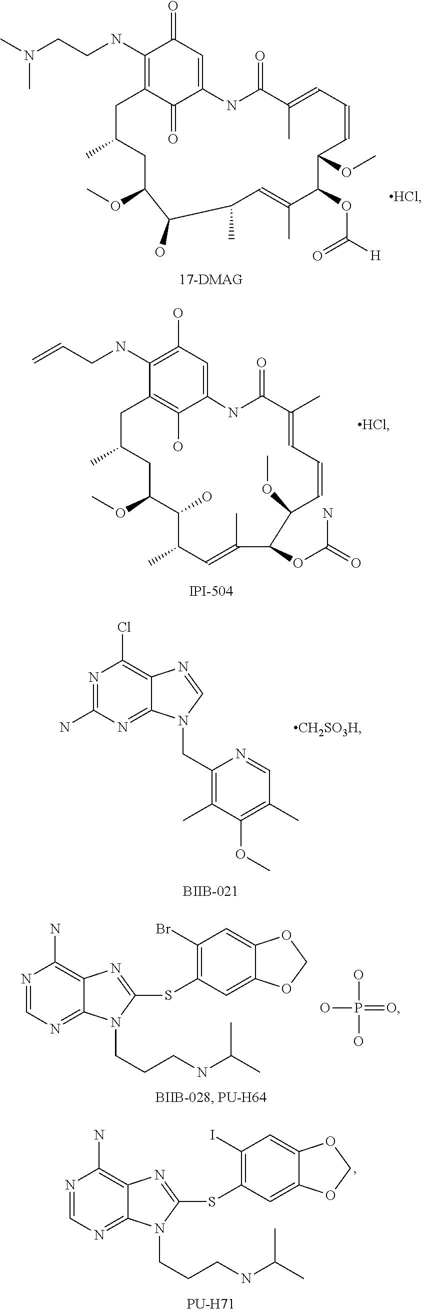

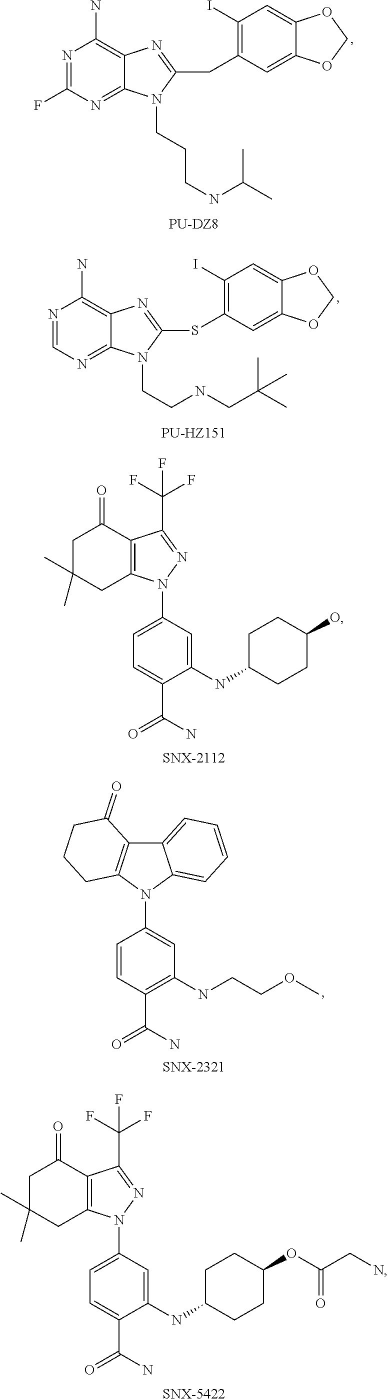

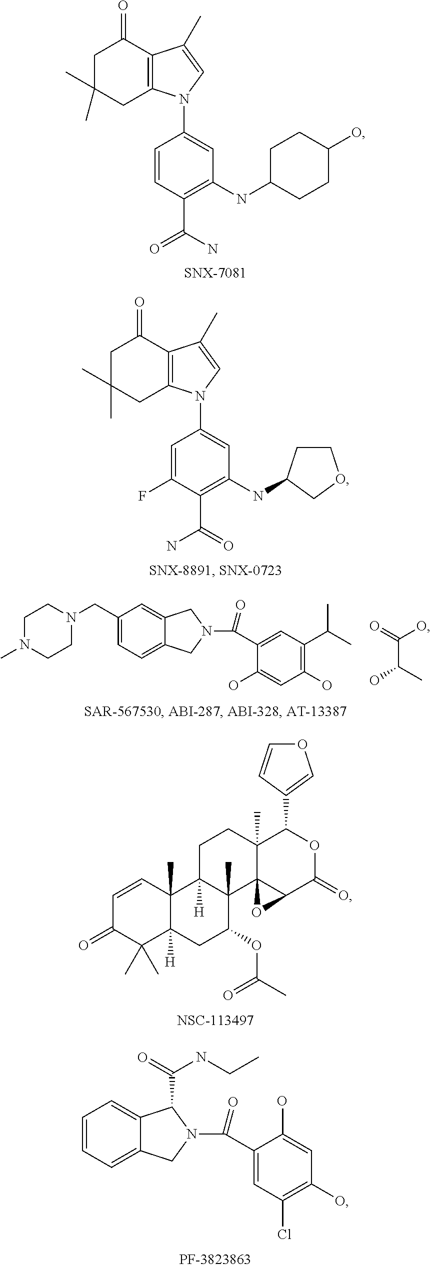

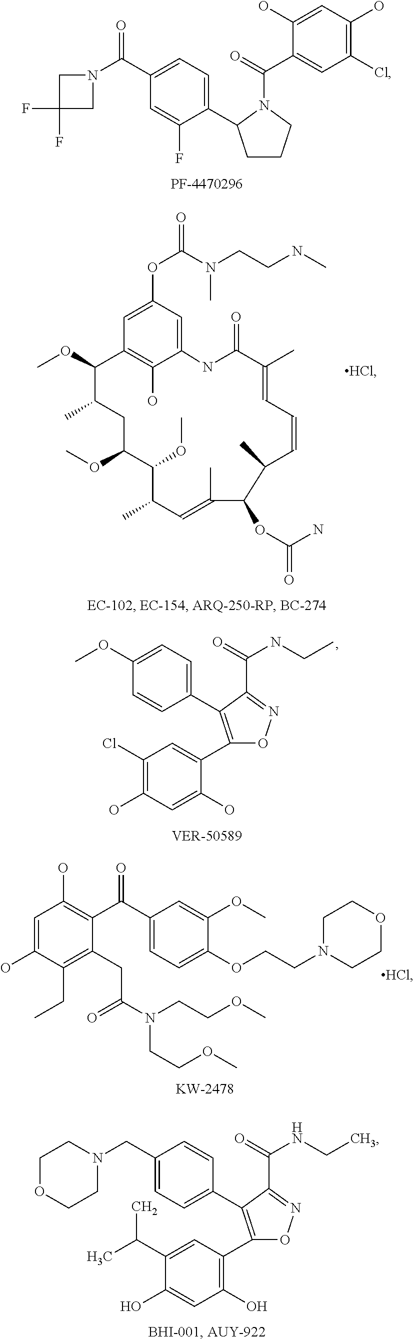

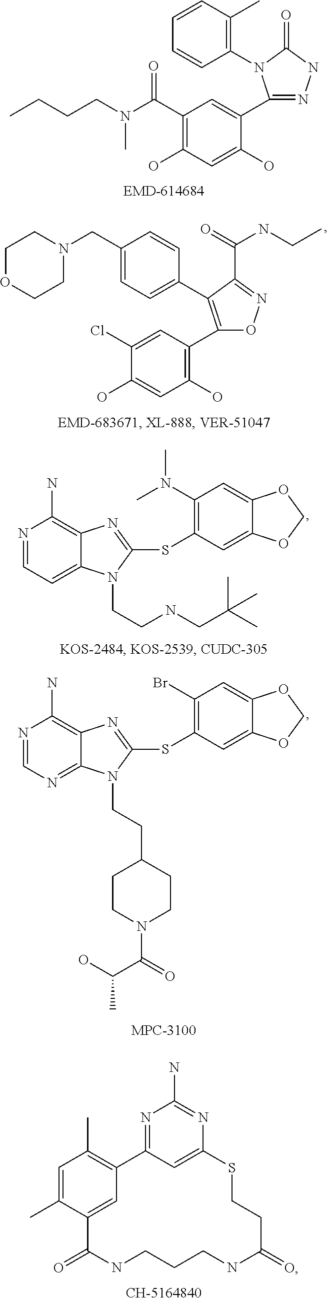

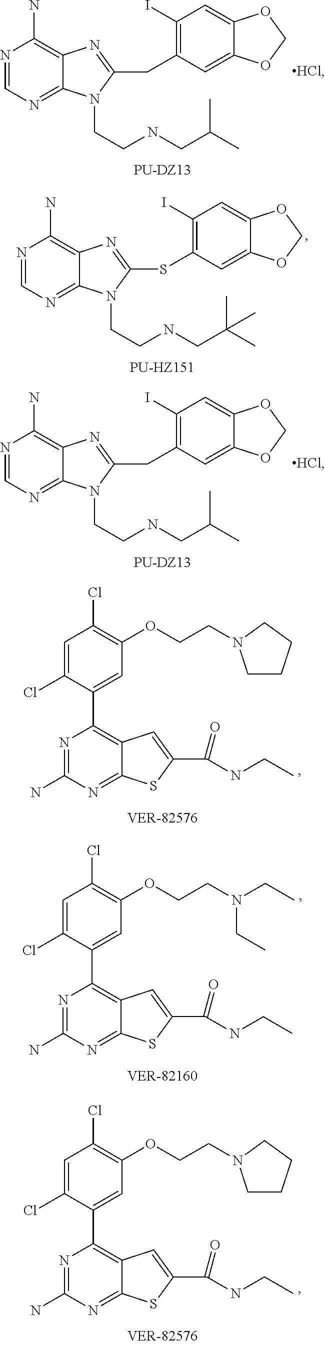 [SMILES: CC(C)CNCCn1c(Cc2cc3c(cc2I)OCO3)nc2c(N)ncnc21 ; CC(C)(C)CNCCn1c(Sc2cc3c(cc2I)OCO3)nc2c(N)ncnc21 ; CC(C)CNCCn1c(Cc2cc3c(cc2I)OCO3)nc2c(N)ncnc21 ; CCNC(=O)c1cc2c(-c3cc(OCCN4CCCC4)c(Cl)cc3Cl)nc(N)nc2s1 ; CCNC(=O)c1cc2c(-c3cc(OCCN(CC)CC)c(Cl)cc3Cl)nc(N)nc2s1 ; CCNC(=O)c1cc2c(-c3cc(OCCN4CCCC4)c(Cl)cc3Cl)nc(N)nc2s1]

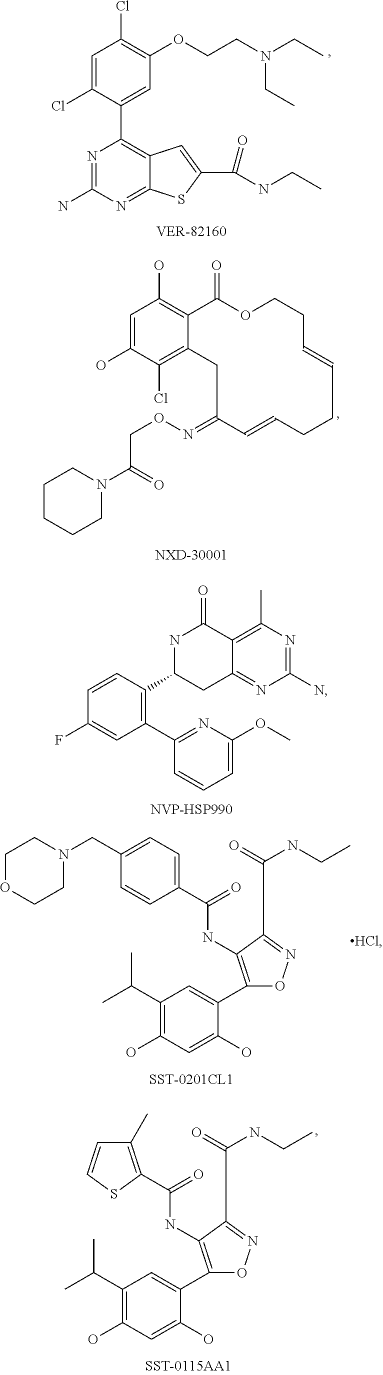

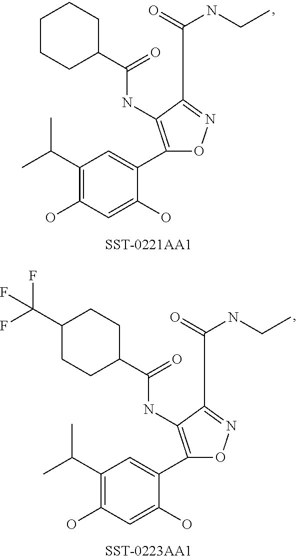 [SMILES: CCNC(=O)c1noc(-c2cc(C(C)C)c(O)cc2O)c1NC(=O)C1CCCCC1 ; CCNC(=O)c1noc(-c2cc(C(C)C)c(O)cc2O)c1NC(=O)C1CCC(C(F)(F)F)CC1]

and novobiocin (a C-terminal Hsp90i). In certain embodiments, the HSP90 inhibitor may be ganetespib, geldamycin and its derivatives (e.g. 17-allyamino-geldanamycin, i.e., tanespimycin, and 17-Dimethylaminoethylamino-17-demethoxygeldanamycin, i.e., alvespimycin), NVP-AUY922 (VER-52296), AT13387, BIIB021, MPC-3100, NVP-BEP800, SNX-2112, PF-04929113 (SNX-5422) herbinmycin A, radicicol, CCT018059, PU-H71, and celastrol. In certain embodiments, the agent is ganetespib. In certain embodiments, the Hsp90 inhibitor is not ganetespib.

The invention further provides kits to practice the methods or uses of diagnosis, treatment, or any other method or use provided herein.

In certain embodiments, the kit includes an Hsp90 inhibitor and instruction for administration of an Hsp90 inhibitor to a subject having a tumor with a high level of hypoxia.

In certain embodiments, the kit includes at least one reagent specifically for detection of a level of hypoxia and instructions for administering an Hsp90 inhibitor to a subject with cancer identified as having a high level of hypoxia. It is understood that not all of the components of the kit need to be in a single package.

In certain embodiments, the Hsp90 inhibitor may be ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIlB-021, BIlB-028, PU-H64, 20 PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, or celastrol. In certain embodiments, the Hsp90 inhibitor is not ganetespib.

More embodiments of the invention are provided infra.

### BRIEF DESCRIPTION OF THE DRAWINGS

FIG. 1 shows the activity of various chemotherapeutic agents in a 72 hr viability assay using MDA-MB-231 breast cancer cells.

FIG. 2 shows the activity of ganetespib in a 24 hr viability assay using SUM149 inflammatory breast cancer (IBC) cells.

FIG. 3 shows the activity of ganetespib in a viability assay in BT-474 breast cancer cells grown as mammospheres in Matrigel®. The cells were treated for 72 hr and analyzed by microscopy. IC50 was determined by AlamarBlue®.

FIG. 4A shows the activity of ganetespib in a single agent viability assay Detroit562 cells, a head and neck cancer cell line, exposed to various chemotherapeutic agents for 72 hr (left).

FIG. 4B shows the expression of various Hsp90 client proteins as determined by western blot of cell extracts from Detroit562 cells exposed to ganetespib for 24 hr (right).

FIG. 5 shows a western blot of protein expression in cell extracts from Detroit 562 head and neck cancer cells treated with 100 nM of ganetespib 24 hours prior to receiving the DNA damaging agent bleomycin (5 μM). Protein expression was measured at the indicated time points after bleomycin treatment. Bleomycin increased both Chk1 and Chk2 phosphorylation, which was blocked when cells were treated first with ganetespib.

FIG. 6 is a waterfall diagram showing the best percentage changes in size of target lesions responses according to ALK status after treatment with ganetespib. The y axis represents the percentage tumor volume change from baseline. For each patient (each bar) the percentage change in measurable tumor at best response was displayed by the genotype of the patient, i.e., ALK status. A subject was considered to be ALK+ (i.e., have an ALK mutation) if a mutation in ALK was detected using any of the methods.

FIG. 7 shows a western blot of Hsp90 client proteins in BT-474 cells after treatment with ganetespib for 16 hours.

FIG. 8 shows a graph of the average tumor volume over time in an MDA-MB-231 xenograft model in response to treatment with ganetespib.

FIG. 9 is a waterfall diagram showing the best response in patients with metastatic breast cancer based on ER, PR, and HER2 marker status in a Phase II clinical trial of ganetespib.

FIG. 10 shows a PET/CT scan of the lungs and bone before and after 19 days of treatment with ganetespib in a female patient with metastatic triple negative breast cancer. Arrows indicate the tumor mass in the lung.

FIG. 11 shows a table of IC50 values for ganetespib in NSCLC cell lines with a KRAS mutation after treatment with ganetespib for 72 hr.

FIG. 12 shows a graph of the results of treatment of various NSCLC cell lines with ganetespib, camptothecin, or a combination thereof for 72 hours.

FIG. 13 shows a graph of the results of treatment of various NSCLC cell lines with ganetespib, pemetrexed, or a combination thereof for 72 hours.

FIG. 14 shows a graph of the results of treatment of various NSCLC cell lines with ganetespib, gemcitabine, or a combination thereof for 72 hours.

FIG. 15 shows a graph of the results of treatment of various NSCLC cell lines with ganetespib, certain platins, or a combination thereof for 72 hours.

FIG. 16 shows a graph of the results of treatment of various NSCLC cell lines with ganetespib, SN-38, or a combination thereof for 72 hours.

FIG. 17 shows a graph of the results of treatment of various NSCLC cell lines with ganetespib, docetaxel, or a combination thereof for 72 hours.

FIG. 18 shows a graph of the results of treatment of various NSCLC cell lines with ganetespib, AZD6244, or a combination thereof for 72 hours.

FIG. 19 shows a graph of the results of treatment of various NSCLC cell lines with ganetespib, BEZ235, or a combination thereof for 72 hours.

FIG. 20 shows a graph of the results of treatment of mice with A549 NSCLC xenografts with ganetespib, BEZ-235, or a combination thereof.

FIGS. 21A and B show the activity of LDH5 as a percent of total LDH activity in serum samples from nude mice with (A) HCT116 tumors or (B) 786-0 tumors relative to tumor volume. FIGS. 21C and D show the protein levels of LDH5 as a percent of total LDH activity in serum samples from nude mice with (C) HCT116 tumors or (D) 786-0 tumors relative to tumor volume.

FIG. 22 shows treatment with ganetespib for 24 hours decreases proliferation of Mia-PaCa2, HPAC and PANC-1 cells (p<0.001, one way ANOVA). These results were further confirmed by XTT assay.

FIG. 23 shows Western blot for Mia-PaCa2, PANC-1 and HPAC cell lines treated with ganetespib for 24 hours. Results indicate decreased levels of HIF-1α a and VEGF levels in pancreatic cancer cell lines.

FIG. 24 shows ELISA assay demonstrates significant (p<0.001, one way ANOVA) down-regulation of VEGF secretion after treatment with ganetespib.

FIG. 25 shows Egg CAM assay-treatment with ganetespib for 24 hours in conditioned medium in three pancreatic cell lines. The conditioned medium was collected from control and treated cells. 100 μl of conditioned medium, either control or treated, was injected into fertilized chicken eggs. Eggs were incubated at 37° C. for 15 days, then dissected and the membrane was photographed.

FIG. 26 shows treatment with ganetespib significantly inhibits tumor growth and decreases angiogenesis in in vivo models of pancreatic cancer.

### DETAILED DESCRIPTION OF THE INVENTION

Research has provided the physician with ever more options for therapeutics for the treatment of cancer. However, despite the availability of the new agents, the ability to match a therapeutic agent to a specific patient based not just on the type of tumor or site of the tumor, but the characteristic of the tumor, is lacking. The instant invention provides methods of identifying a subject who will likely respond favorably to treatment with an Hsp90 inhibitor by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, fecal matter, circulating tumor cells, bronchial lavage, peritoneal lavage, exudate, effusion, and sputum for the presence of one or more indicators of the level of hypoxia in the tumor.

Serum LDH level is well established as a prognostic factor associated with poor outcomes and large tumor burden in many tumor types. It is, therefore, interesting to note that a number of reports from large randomized phase 2 and phase 3 studies for several anti-cancer agents have shown a positive interaction between clinical outcomes and high baseline LDH levels. These include bevacizumab in pancreatic cancer (high LDH: OS HR=0.59, 95% CI 0.43-0.82; normal LDH: HR=0.98, 95% CI 0.78-1.24); bevacizumab in prostate cancer (high LDH: OS HR=0.80, P=0.029; normal LDH: OS HR=1.02, P=0.87); bevacizumab in melanoma (high LDH: OS HR=0.53, 95% CI 0.32-0.88; normal LDH: OS HR=1.25, 95% CI 0.73-1.25); temsirolimus in RCC (high LDH: OS HR=0.56, P=0.002; normal LDH: OS HR=0.90, P=0.51); vatalanib in colon cancer first line (high LDH: PFS HR=0.67, P=0.009; PFS HR=0.88, P=0.188); and vatalanib in colon cancer second line (high LDH: PFS HR=0.63, P<0.001; PFS HR=0.83, P=0.01).

The VEGF and mTOR signaling pathways are regulated by hypoxia, both at the transcriptional and translational level. The oxygen-sensitive transcription factor HIF-1α a is one of the principal mediators of the hypoxic response in cancer cells, including the metabolic switch from oxidative phosphorylation to glycolysis. The hypoxia-regulated LDH A gene is under transcriptional control of HIF-1. Therefore, serum LDH levels may in part reflect tumor oxygenation and metabolic status. This connection between tumor oxygenation and serum LDH levels may explain the enhanced activity seen in patients with high serum LDH levels for drugs that affect hypoxia-mediated signaling pathways, such as VEGF and mTOR inhibitors.

There is therefore evidence that both VEGF/mTOR inhibitors are sensitive to tumor oxygenation and metabolic status. Both classes of drugs seem to preferentially work in anaerobic tumor cells.

Hsp90 inhibitors effect on hypoxia-driven pathways, including VEGF and mTOR. For example, Hsp90 inhibitors inhibit HIF1-α. Further, several key elements of the VEGF and mTOR pathways are client proteins VEGF, VEGFR1-3, IGF-1R, GLUT1-3, PI3K of Hsp90. As demonstrated herein, ganetespib down-regulates the expression or phosphorylation of Hsp90 client proteins. Therefore, Hsp90 inhibitors should be useful in the treatment of subjects with cancer wherein the tumor has a high level of hypoxia.

In order that the present invention may be more readily understood, certain terms are first defined. In addition, it should be noted that whenever a value or range of values of a parameter are recited, it is intended that values and ranges intermediate to the recited values are also intended to be part of this invention.

### I. DEFINITIONS

The articles “a”, “an” and “the” are used herein to refer to one or to more than one (i.e., to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations.

As used herein, an “Hsp90 inhibitor” is understood as a therapeutic agent that reduces the activity of Hsp90 either by directly interacting with Hsp90 or by preventing the formation of the Hsp90/CDC37 complex such that the expression and proper folding of at least one client protein of Hsp90 is inhibited. “Hsp90” includes each member of the family of heat shock proteins having a mass of about 90-kilodaltons. For example, in humans the highly conserved Hsp90 family includes cytosolic Hsp90 and Hsp90 isoforms, as well as GRP94, which is found in the endoplasmic reticulum, and HSP75/TRAP1, which is found in the mitochondrial matrix. As used herein, Hsp90 inhibitors include, but are not limited to ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIlB-021, BIlB-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, and celastrol. In certain embodiments, Hsp90 inhibitors do not include ganetespib.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that identifying a subject or selecting a subject as having a specific level of hypoxia or a specific level of LDH can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject e.g., by an identification card, hospital bracelet, asking the subject for his/her name and/or other personal information to confirm the subject's identity.

As used herein, the term “benefit” refers to something that is advantageous or good, or an advantage. Similarly, the term “benefiting”, as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, me dulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. In certain embodiments, the sample can be the portion of the subject that is imaged (e.g., using a PET scan, a functional imaging method such as MRI to detect blood flow) or tested to determine level of hypoxia (e.g., tumor tissue assayed for level of hypoxia using a probe). Samples are typically removed from the subject prior to analysis, however, tumor samples can be analyzed in the subject, for example, using imaging or other detection methods.

In some embodiments, only a portion of the sample is subjected to an assay for determining the level of hypoxia or the level of the tumor using any method provided herein. In certain embodiments, the level of hypoxia is indicated by the level of an isoform or subunit of lactate dehydrogenase (LDH) or any combination of subunits or isoforms including total LDH, or various portions of the sample are subjected to various assays for determining the level of hypoxia or the level of an isoform or subunit of LDH. Also, in many embodiments, the sample may be pre-treated by physical or chemical means prior to the assay. For example, samples, e.g., blood samples, can be subjected to centrifugation, dilution and/or treatment with a solubilizing substance prior to assaying the samples for the level of hypoxia or LDH. Such techniques serve to enhance the accuracy, reliability and reproducibility of the assays of the present invention.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of LDH in a control sample that consists of a group of measurements may be determined, e.g., based on any appropriate statistical measure, such as, for example, measures of central tendency including average, median, or modal values.

The term “control level” refers to an accepted or pre-determined level of hypoxia or LDH which is used to compare with the level of hypoxia or LDH in a sample derived from a subject. For example, in one embodiment, the control level of hypoxia is based on the level of hypoxia in sample(s) from a subject(s) having slow disease progression. In another embodiment, the control level of hypoxia is based on the level in a sample from a subject(s) having rapid disease progression. In another embodiment, the control level of hypoxia is based on the level of hypoxia in a sample(s) from an unaffected, i.e., non-diseased, subject(s), i.e., a subject who does not have cancer. In yet another embodiment, the control level of hypoxia is based on the level of hypoxia in a sample from a subject(s) prior to the administration of a therapy for cancer. In another embodiment, the control level of hypoxia is based on the level of hypoxia in a sample(s) from a subject(s) having cancer that is not contacted with a test compound. In another embodiment, the control level of hypoxia is based on the level of hypoxia in a sample(s) from a subject(s) not having cancer that is contacted with a test compound. In one embodiment, the control level of hypoxia is based on the level of hypoxia in a sample(s) from an animal model of cancer, a cell, or a cell line derived from the animal model of cancer. In another embodiment, the control level of hypoxia is listed in a chart.

In one embodiment, the control is a standardized control, such as, for example, a control which is predetermined using an average of the levels of hypoxia from a population of subjects having no cancer. In still other embodiments of the invention, a control level of hypoxia is based on the level of hypoxia in a non-cancerous sample(s) derived from the subject having cancer. For example, when a biopsy or other medical procedure reveals the presence of cancer in one portion of the tissue, the control level of hypoxia may be determined using the non-affected portion of the tissue, and this control level may be compared with the level of hypoxia in an affected portion of the tissue. Similarly, when a biopsy or other medical procedure reveals the presence of a cancer in one portion of the tissue, the control level of hypoxia may be determined using the non-affected portion of the tissue, and this control level may be compared with the level of hypoxia in an affected portion of the tissue.

As used herein, the term “obtaining” is understood herein as manufacturing, purchasing, or otherwise coming into possession of.

As used herein, the term “lactate dehydrogenase” refers to an enzyme that interconverts pyruvate and lactate with concomitant interconversion of NADH and NAD+. Under conditions of hypoxia, the reaction favors the conversion of pyruvate to lactate. Under conditions of normoxia, or low levels of hypoxia, the reaction favors the conversion of lactate to pyruvate. Functional lactate dehydrogenase are homo- or heterotetramers composed of M and H protein subunits encoded by the LDHA and LDHB genes respectively: LDH-1 (4H) is the predominant form found, for example, in the heart and red blood cells (RBCs); LDH-2 (3H1M) is the predominant found, for example, in the reticuloendothelial system; LDH-3 (2H2M) is the predominant form found, for example, in the lungs; LDH-4 (1H3M) is the predominant form found, for example, in the kidneys, placenta and pancreas; and LDH-5 (4M) is the predominant form found, for example, in the liver and striated muscle. Typically, multiple forms of LDH are found in these tissues. Lactate dehydrogenase is classified as (EC 1.1.1.27). The specific ratios tested may be tumor-type specific.

As used herein, the terms “hypoxia” and “hypoxic” refer to a condition in which a cancer or a tumor has a low oxygen microenvironment or a less well-oxygenated microenvironment. Hypoxia occurs when tumor growth exceeds new blood vessel formation, and a tumor must undergo genetic and adaptive changes to allow them to survive and proliferate in the hypoxic environment. The development of intratumoral hypoxia is a common sign of solid tumors. When a tumor microenvironment is less well-oxygenated, there is a greater dependency on oxygen-sensitive pathways, including but not limited to HIF1α pathways, VEGF pathways, and mTOR pathways. These pathways facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002). These pathways may also facilitate invasion and metastasis. Accordingly, the treatment of a subject with a cancer or tumor with a selected agent such as bevacizumab, ganetespib, temsirolimus, erlotinib, PTK787, BEZ235, XL765, pazopanib, cediranib, or axitinib is more effective when the subject has a tumor that exhibits a modulated level of hypoxia, e.g., a high level of hypoxia. As the level of hypoxia in the tumor can be determined by obtaining a sample from a site other than the tumor, as used herein, the subject can be stated to demonstrate a modulated level of hypoxia when it is the tumor present in the subject that demonstrates a modulated level of hypoxia. As used herein it is understood that the subject with a modulated level of hypoxia is typically not suffering from systemic oxygen imbalance or ischemic disease at a site remote from the tumor.

As used herein, the term “level of hypoxia” is understood as the amount of one or more markers indicative of a low oxygen level, or cells having characteristics and/or employing biological pathways characteristic of cells with a low oxygen level, e.g., due to the Warburg effect. Such markers include, but are not limited to, lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), and ornithine decarboxylase (ODC). Tumor size can also be correlated with a level of hypoxia. A level of hypoxia can also be determined by PET scan. LDH can be one or more isoforms or subunits of LDH such as LDH5, LDH4, LDH3, LDH2, LDH1, LDHM (also known as LDHA) and LDHH (also known as LDHB). In one embodiment, LDH can be a total sample of all LDH isoforms or subunits. “Hypoxia inducible factors” or “HIFs” are transcription factors which respond to changes in available oxygen in a cellular environment. HIF1α is a master regulator of hypoxic gene expression and oxygen homeostasis. HIF can be one or more subunits or isoforms of HIF including HIF-1α, HIF-1β, HIF-2α, and HIF-2β. VEGF can be one or more of the various splice forms of VEGF including pro-angiogenic VEGF-A and antiangiogenic VEGF-B.

As used herein, the term “level of LDH” refers to the amount of LDH present in a sample which can be used to indicate the presence or absence of hypoxia in the tumor in the subject from whom the sample was obtained. LDH enables the conversion of pyruvate to lactate and is a critical component of glycolysis under hypoxic conditions. LDH can be total LDH or one or more isoforms or subunits of LDH such as LDH5, LDH4, LDH3, LDH2, LDH1, LDHM (also known as LDHA) and LDHH (also known as LDHB). A modulated level of LDH can refer to a high level of LDH or a low level of LDH. In one embodiment, a PET scan (which is positive when aerobic glycolysis is active) is an indicator of a high level of LDH. In another embodiment, a PET scan (which is negative when aerobic glycolysis is inactive) is an indicator of a low level of LDH. In one embodiment, a high level of LDH is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normal level of LDH. In another embodiment, a low level of LDH is 0.9, 0.8, 0.7, 0.6, 0.5, 0.4, 0.3, 0.2, or 0.1 times the value of a normal level of LDH. A normal level of LDH, or any other marker, can be defined as any value within the range of normal, or the upper limit of the normal value, or the lower limit of the normal value. Assays for determining the level of LDH in a sample are well known in the art and provided herein.

In another embodiment, the level of LDH can be understood to be a change in the relative levels of protein or activity of LDH isoforms or the ratio of LDH isoforms. Preferably, the ratios are the ratios of normalized values, e.g., the level of the LDH subunit or isoform is normalized to the ULN, the LLN, or a median value. A change of the relative levels of the isoforms can be indicative of the level of hypoxia. For example, an increase in the level of LDHA relative to LDHB can be indicative of an increase in hypoxia. Alternatively, an increase in the level of LDH5 and/or LDH4, either individually or in total, relative to the level of LDH1 or total LDH can be indicative of an increase in hypoxia. The relative levels can be compared to relative levels in an appropriate control sample from normal subjects, e.g., subjects without cancer or ischemic disease. That is, the ratios are the ratios of normalized values, e.g., the level of the LDH subunit or isoform is normalized to the ULN, the LLN, or a median value. The normal levels can be considered to be a range with an upper level of normal and a lower level of normal. In certain embodiments, a high level of LDH can be understood an increase in the normalized level of LDHA or LDH5 and/or LDH4 relative to the normalized level of LDHB or LDH1 or total LDH, respectively, or to total LDH of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normalized level of LDHA or LDH5 and/or LDH4 relative to the normalized level of LDHB or LDH1 or total LDH, respectively. In another embodiment, a low level of LDH is a ratio of 0.9, 0.8, 0.7, 0.6, 0.5, 0.4, 0.3, 0.2, or 0.1 of the normalized value of LDHA or LDH5 and/or LDH4 relative to the normalized level of LDHB or LDH1 or total LDH, respectively.

As used herein, a “normalized ratio” is understood as a proportion of two values that have been compared to a standard, either an external (e.g., population control level) or an internal (e.g., level from a normal tissue, level from an earlier time point, level of one or more isoforms) control to allow for comparison of samples between individuals. For example, the ratio of normalized levels of hypoxia-modulated polypeptides can be determined by determining a ratio of two normalized levels of two isoforms or subunits of LDH or total LDH by comparing the level of a first isoform or subunit of LDH in the sample relative to a control sample to provide a first normalized level, and the level of a second isoform or subunit of LDH or total LDH relative to a control sample to provide a second normalized level, and calculating a ratio of the first normalized level and the second normalized level to provide a normalized ratio of LDH isoforms or subunits, wherein at least one of the first level and the second level are not total LDH. In certain embodiments, a low level of hypoxia is a normalized ratio of the ULN of LDHA to LDHB of 1.0 or less, or a normalized ratio of the ULN of LDH5 and/or LDH4 to LDH1 or total LDH of 1.0 or less.

Assays for determining the level of LDH in a sample are well known in the art. See, e.g., U.S. Publication Nos. 2010/0178283 and 2008/0213744 and U.S. Pat. Nos. 4,250,255 and 6,242,208, the entire contents of each of which are expressly incorporated herein by reference. LDH sequences are further provided in public databases (e.g., at http://blast.ncbi.nlm.nih.gov/Blast.cgi).

It is also understood that levels of the various markers can include the level of a post-translationally modified marker, e.g., the total amount of an isoform of HIF may remain the same, but the amount of the hydroxylated version of the HIF may increase. In addition, it is noted that HIF and other hypoxia-modulated polypeptides can be up-regulated by a number of conditions other than hypoxia, e.g., pH change, changes in levels of O₂.^{•} or H₂O₂, etc. Accordingly, although the term “level of expression,” as used herein, is intended to encompass all hypoxia responsive factors, a change in their level of expression may or may not actually directly reflect the amount of oxygen available to the tumor.

Methods to detect the levels of markers of hypoxia are well known in the art. Antibodies against and kits for detection of hypoxia-modulated polypeptides can be purchased from a number of commercial sources. Alternatively, using routine methods known in the art (e.g., immunization of animals, phage display, etc.) antibodies against one or more hypoxia-modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Hypoxia can be detected by enzyme activity assays (e.g., LDH activity, kinase activity) including in gel assays to resolve the activity of various isoforms of proteins. Alternatively, immunohistochemical methods can be used on tumor samples and tissue sections. Antibodies against prodrugs that localize in hypoxic regions (e.g., EF5, pimonidazole, etc.) can also be used to detect hypoxia. Functional imaging measuring blood flow in the tumor can be used as an indicator of hypoxia in the tissue. Direct measurement of hypoxia can be performed by inserting a sensor into the tumor. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample.

Alternatively, nucleic acid-based methods of detection of levels of hypoxia are also well known in the art. Methods of designing primers and probes for quantitative reverse transcription real time (rt) PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample.

“Baseline” refers to the level of hypoxia or the level of LDH upon patient entrance into the study and is used to distinguish from levels of hypoxia or levels of LDH the patient might have during or after treatment.

“Elevated” or “lower” refers to a patient's value relative to the upper limit of normal (“ULN”) or the lower limit of normal (“LLN”) which are based on historical normal control samples. As the level of the hypoxic marker present in the subject will be a result of the disease, and not a result of treatment, typically a control sample obtained from the patient prior to onset of the disease will not likely be available. Because different labs may have different absolute results, LDH values are presented relative to that lab's upper limit of normal value (ULN). LDH can be expressed in IU/ml (International Units per milliliter). An accepted ULN for LDH is 234 IU/ml, however, this value is not universally accepted or applicable to all methods of detection of LDH in all samples.

The specific value for ULN and LLN will also depend, for example, on the type of assay (e.g., ELISA, enzyme activity, immunohistochemistry, imaging), the sample to be tested (e.g., serum, tumor tissue, urine), and other considerations known to those of skill in the art. The ULN or LLN can be used to define cut-offs between normal and abnormal. For example, a low level of a marker (e.g., LDH) can be defined as a marker level less than or equal to the ULN for that marker, with a high level being all values greater than the ULN. Cut-offs can also be defined as fractional amounts of the ULN. For example, a low level of a marker can be understood to be a level of about 0.5 ULN or less, 0.6 ULN or less, 0.7 ULN or less, 0.8 ULN or less, 0.9 ULN or less, 1.0 ULN or less, 1.1 ULN or less, 1.2 ULN or less, 1.3 ULN or less, 1.4 ULN or less, 1.5 ULN or less, 1.6 ULN or less, 1.7 ULN or less, 1.8 ULN or less, 1.9 ULN or less, 2.0 ULN or less, 2.5 ULN or less, 3.0 ULN or less, or 4.0 ULN or less, with the corresponding high level of the marker being a value greater than the low level. In certain embodiments, the presence of a low level of a marker in a subject sample as defined above can be indicative that a subject will or will not respond to a particular therapeutic intervention. In certain embodiments, the presence of a high level of a marker in a subject sample as defined above can be indicative that a subject will or will not respond to a particular therapeutic intervention.

Marker levels can also be further stratified, for example, into low, intermediate, and high based on the ULN value. For example, the presence of a low level of a marker in a subject sample as defined above can be indicative that a subject will or will not respond to a particular therapeutic intervention. An intermediate level of a marker, e.g., a range bracketed by any range within the values of 0.5 ULN, 0.6 ULN, 0.7 ULN, 0.8 ULN, 0.9 ULN, 1.0 ULN, 1.1 ULN, 1.2 ULN, 1.3 ULN, 1.4 ULN, 1.5 ULN, 1.6 ULN, 1.7 ULN, 1.8 ULN, 1.9 ULN, and 2.0 ULN, can be considered an intermediate range wherein the level of the marker may be indeterminate that a subject will or will not respond to a particular therapeutic intervention. A high level, greater than the intermediate level, would be indicative that a subject will or will not respond to a particular therapeutic intervention.

Similarly, cut-offs of ratios of LDH subunits or isoforms comparing the ULN, the LLN, or the median values to differentiate between high and low levels of hypoxia can be defined as any value or range bracketed by the values 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, or higher.

The “normal” level of expression of a marker is the level of expression of the marker in cells of a subject or patient not afflicted with cancer. In one embodiment, a “normal” level of expression refers to the level of expression of the marker under normoxic conditions.

An “over-expression” or “high level of expression” of a marker refers to an expression level in a test sample that is greater than the standard error of the assay employed to assess expression, and is preferably at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of the marker in a control sample (e.g., sample from a healthy subject not having the marker associated disease, i.e., cancer). In one embodiment, expression of a marker is compared to an average expression level of the marker in several control samples.

A “low level of expression” or “under-expression” of a marker refers to an expression level in a test sample that is less than at least 0.9, 0.8, 0.7, 0.6, 0.5, 0.4, 0.3, 0.2, or 0.1 times the expression level of the marker in a control sample (e.g., sample from a healthy subjects not having the marker associated disease, i.e., cancer). In one embodiment, expression of a marker is compared to an average expression level of the marker in several control samples.

As used herein, the term “identical” or “identity” is used herein in relation to amino acid or nucleic acid sequences refers to any gene or protein sequence that bears at least 30% identity, more preferably 40%, 50%, 60%, 70%, 75%, 80%, 81%, 82%, 83%, 84%, 85%, 86%, 87%, 88%, 89%, 90%, 91%, 92%, 93%, 94%, and most preferably 95%, 96%, 97%, 98%, 99% or more identity to a known gene or protein sequence over the length of the comparison sequence. Protein or nucleic acid sequences with high levels of identity throughout the sequence can be said to be homologous. A “homologous” protein can also have at least one biological activity of the comparison protein. In general, for proteins, the length of comparison sequences will be at least 10 amino acids, preferably 10, 20, 30, 40, 50, 60, 70, 80, 90, 100, 150, 175, 200, 250, or at least 300 amino acids or more. For nucleic acids, the length of comparison sequences will generally be at least 25, 50, 100, 125, 150, 200, 250, 300, 350, 400, 450, 500, 550, 600, 650, 700, 800, or at least 850 nucleotides or more.

By “hybridize” is meant pairing to form a double-stranded molecule between complementary polynucleotide sequences, or portions thereof, under various conditions of stringency. (See, e.g., Wahl and Berger Methods Enzymol. 152:399, 1987; Kimmel, Methods Enzymol. 152:507, 1987.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

As used herein, the term “oxygen-sensitive pathway” is a cellular signaling pathway which is activated by hypoxia. Oxygen-sensitive pathways may be up-regulated by hypoxia. Alternatively, an oxygen-sensitive pathway may be down-regulated by hypoxia. Oxygen-sensitive pathways include, but are not limited to, HIF pathways (such as HIF1α pathways), VEGF pathways, and mTOR pathways. As used herein, the term “hypoxia-modulated gene” or “hypoxia-modulated polypeptide” refers to a gene or protein which is up-regulated or down-regulated by hypoxia.

As used herein, the term “HIF pathway” and “HIF pathway members” as used herein, describe proteins and other signaling molecules that are regulated by HIF-1 and HIF-2. Hypoxia-Inducible Factor 1 (HIF-1) is a transcription factor that has been shown to play an essential role in cellular responses to hypoxia. Upon hypoxic stimulation, HIF-1 has been shown to activate genes that contain Hypoxic Response Elements (HREs) in their promoters, and thus up-regulate a series of gene products that promote cell survival under conditions of low oxygen availability. The list of known HIF-responsive genes includes glycolytic enzymes (such as lactate dehydrogenase (LDH), enolase-1 (ENO-I), and aldolase A, glucose transporters (GLUT 1 and GLUT 3), vascular endothelial growth factor (VEGF), inducible nitric oxide synthase (NOS-2), and erythropoietin (EPO). The switch of the cell to anaerobic glycolysis, and the up-regulation of angiogenesis by VEGF is geared at maximizing cell survival under conditions of low oxygen tension by reducing the requirement for oxygen, and increasing vasculature to maximize oxygen delivery to tissues. The HIF-1 transcription complex has recently been shown to comprise a heterodimer of two basic helix-loop-helix proteins, HIF-1α a and HIF-1β (also known as ARNT, Aryl Hydrocarbon Receptor Nuclear Translocator).

HIF-1α a is a member of the basic-helix-loop-helix PAS domain protein family and is an approximately 120 kDa protein containing two transactivation domains (TAD) in its carboxy-terminal half and DNA binding activity located in the N-terminal half of the molecule. HIF-1α a is constitutively degraded by the ubiquitin-proteosome pathway under conditions of normoxia, a process that is facilitated by binding of the von Hippel-Lindau (VHL) tumor suppressor protein to HIF-la. Under conditions of hypoxia, degradation of HIF-1α a is blocked and active HIF-1α a accumulates. The subsequent dimerization of HIF-la with ARNT leads to the formation of active HIF transcription complexes in the nucleus, which can bind to and activate HREs on HIF-responsive genes.

As used herein, the term “VEGF pathway” and “VEGF pathway members” as used herein, describe proteins and other signaling molecules that are regulated by VEGF. For example, VEGF pathway members include VEGFR1, 2, and 3; PECAM-1, LacCer synthase, and PLA2.

As used herein, the term “mTOR pathway” and “mTOR pathway members” as used herein, describe proteins and other signaling molecules that are regulated by mTOR. For example, mTOR pathway members include SK6, PDCD4, eIF4B, RPS6, eIF4, 4E-BP1, and eIF4E.

“Chemotherapeutic agent” is understood as a drug used for the treatment of cancer. Chemotherapeutic agents include, but are not limited to, small molecules and biologics (e.g., antibodies, peptide drugs, nucleic acid drugs). In certain embodiments, a chemotherapeutic agent does not include one or more of bevacizumab, ganetespib, temsirolimus, erlotinib, PTK787, BEZ235, XL765, pazopanib, cediranib, and axitinib.

As used herein, an “Hsp90 inhibitor” is one or more of ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIlB-021, BIlB-028, PU-H64, 20 PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, and celastrol. In certain embodiments, the selected agent is bevacizumab. In certain embodiments, the selected agent is ganetespib.

As used herein, “detecting”, “detection” and the like are understood that an assay performed for identification of a specific analyte in a sample, e.g., a hypoxia-modulated polypeptide or a hypoxia-modulated gene in a sample. The amount of analyte or activity detected in the sample can be none or below the level of detection of the assay or method.

The terms “modulate” or “modulation” refer to up-regulation (i.e., activation or stimulation), down-regulation (i.e., inhibition or suppression) of a level, or the two in combination or apart. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, “level of activity” is understood as the amount of protein activity, typically enzymatic activity, as determined by a quantitative, semi-quantitative, or qualitative assay. Activity is typically determined by monitoring the amount of product produced in an assay using a substrate that produces a readily detectable product, e.g., colored product, fluorescent product, or radioactive product. For example, the isoforms of LDH in a sample can be resolved using gel electrophoresis. Lactate, nicotinamide adenine dinucleotide (NAD+), nitroblue tetrazolium (NBT), and phenazine methosulphate (PMS) can be added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of each isoform to the other isoforms or total LDH can be determined, for example, by densitometry.

As used herein, “changed as compared to a control” sample or subject is understood as having a level of the analyte or diagnostic or therapeutic indicator (e.g., marker) to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, or a protein) or a substance produced by a reporter construct (e.g., β-galactosidase or luciferase). Depending on the method used for detection the amount and measurement of the change can vary. Changed as compared to a control reference sample can also include a change in one or more signs or symptoms associated with or diagnostic of disease, e.g., cancer. Determination of statistical significance is within the ability of those skilled in the art, e.g., the number of standard deviations from the mean that constitute a positive result.

As used herein, “binding” is understood as having at least a 10² or more, 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

Agents for Treatment of Tumors with High Levels of Hypoxia with Selected Agents

The invention provides methods of use of Hsp90 inhibitors that are more effective in treating disease, e.g., cancer, when administered to a patient with a cancer or tumor exhibiting high levels of hypoxia. In one embodiment, the Hsp90 inhibitor may be ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIlB-021, BIlB-028, PU-H64, 20 PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i), herbinmycin A, radicicol, CCT018059, PU-H71, or celastrol. In an embodiment, the selected agent is ganetespib. In another embodiment, an Hsp90 inhibitor is more effective in treating disease, e.g., cancer, when administered to a patient with a cancer or tumor exhibiting high levels of LDH.

### Ganetespib

Ganetespib (also known as STA-9090) is a Heat Shock Protein 90 (Hsp90) inhibitor having the following structure:

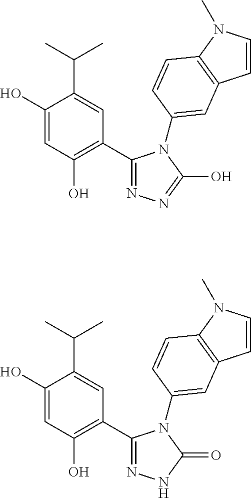 [SMILES: CC(C)c1cc(-c2nnc(O)n2-c2ccc3c(ccn3C)c2)c(O)cc1O ; CC(C)c1cc(-c2n[nH]c(=O)n2-c2ccc3c(ccn3C)c2)c(O)cc1O]

and the chemical name 3-2,4-dihydroxy-5-isopropyl-phenyl)-4-(1-methyl-indol-5-yl)-5-hydroxy-[1,2,4]triazole (see, e.g., U.S. Pat. No. 7,825,148, incorporated herein by reference).

Hsp90 is a chaperone protein required for the proper folding and activation of other cellular proteins, particularly kinases, such as AKT, BCR-ABL, BRAF, KIT, MET, EGFR, FLT3, HER2, PDGFRA and VEGFR. These proteins have been shown to be critical to cancer cell growth, proliferation, and survival. Ganetespib has shown potent activity against a wide range of cancer types, including lung, prostate, colon, breast, gastric, pancreatic, gastrointestinal stromal tumors (GIST), melanoma, AML, chronic myeloid leukemia, Burkitt's lymphoma, diffuse large B-cell lymphoma and multiple myeloma in in vitro and in vivo models. Ganetespib has also shown potent activity against cancers resistant to imatinib, sunitinib, erlotinib and dasatinib.

Ganetespib is more effective in treating disease, e.g., cancer, when administered to a patient with a cancer or tumor exhibiting high levels of hypoxia. In another embodiment, ganetespib is more effective in treating disease, e.g., cancer, when administered to a patient with a cancer or tumor exhibiting high levels of LDH.

### II. COMPOSITIONS, DOSAGES AND MODES OF ADMINISTRATION

In certain embodiments, the invention also provides compositions for treating subjects having cancer, wherein the cancer comprises a tumor with a high level of hypoxia. In certain embodiments, the composition comprising ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIlB-021, BIlB-028, PU-H64, 20 PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i), herbinmycin A, radicicol, CCT018059, PU-H71, and celastrol, In certain embodiments, the composition does not comprise ganetespib.

In certain embodiments, the composition is for treating a subject having a solid tumor. In certain embodiments, the composition is for treating a subject having primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, or refractory malignancy.

In certain embodiments, the composition is for treating a subject with tumor, wherein the level of hypoxia in the tumor is determined in a subject sample. In an embodiment, the subject sample may be tumor tissue, blood, urine, stool, lymph, cerebrospinal fluid, circulating tumor cells, bronchial lavage, peritoneal lavage, exudate, effusion, or sputum. In an embodiment, the tumor tissue is in the subject. In an embodiment, the tumor tissue is removed from the subject.

In certain embodiments, the composition is for treating a subject with tumor, wherein the level of hypoxia in the tumor is determined by detecting the activity level or expression level of one or more hypoxia-modulated polypeptides. In an embodiment, the activity level or expression level of the one or more hypoxia-modulated polypeptides may be up regulated in the sample.

In certain embodiments, the composition is for treating a subject with tumor, wherein the level of hypoxia in the tumor is determined by detecting the activity level or expression level of one or more hypoxia-modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level. In an embodiment, the isoform or subunit of LDH comprises one or more LDH5, LDH4, LDH3, LDH2, LDH1, LDHA and LDHB; or any combination thereof including total LDH. In an embodiment, the isoform of HIF comprises one or more of HIF-1α, HIF-1β, HIF-2α, and HIF-2β; or any combination thereof including total HIF-1 and/or HIF-2. In an embodiment, the pro-angiogenic isoform of VEGF is any VEGF-A isoform, or any combination of VEGF-A isoforms including total VEGF-A.

In certain embodiments, the composition is for treating a subject with tumor, wherein detection of a high level of activity or expression of at least one LDH isoform or subunit in the tumor comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more.

In certain embodiments, the composition is for treating a subject with tumor, wherein detection of a high level of activity or expression of at least one LDH isoform or subunit in the tumor comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, the composition is for treating a subject with tumor, wherein detection of a high level of hypoxia in the tumor comprises detection of a change in a ratio or levels of activity or expression or a change in a ratio of normalized levels of activity or expression of hypoxia-modulated polypeptides. In an embodiment, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the invention also provides compositions for treating subjects having cancer with a high level of hypoxia, wherein the subjects were previously treated with another chemotherapeutic agent. In certain embodiments, the composition comprises an Hsp90 inhibitor, wherein the Hsp90 inhibitor may be ganetespib, geldanamycin (tanespimycin), IPI-493, macbecins, tripterins, tanespimycins, 17-AAG (alvespimycin), KF-SS823, radicicols, KF-S8333, KF-S8332, 17-DMAG, IPI-S04, BIIB-021, BIIB-028, PU-H64, PU-H71, PU-DZ8, PU-HZ1S1, SNX-2112, SNX-2321, SNX-S422, SNX-7081, SNX-8891, SNX-0723, SAR-S67S30, ABI-287, ABI-328, AT-13387, NSC-113497, PF-3823863, PF-4470296, EC-102, EC-154, ARQ-250-RP, BC-274, VER-50589, KW-2478, BHr-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i), herbinmycin A, radicicol, CCT018059, PU-H71, or celastrol. In some embodiments, the composition comprises an Hsp90 inhibitor wherein the Hsp90 inhibitor is not ganetespib.

Techniques and dosages for administration vary depending on the type of compound (e.g., chemical compound, antibody, antisense, or nucleic acid vector) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral, or local by direct injection into the amniotic fluid. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

The composition can be in the form of a pill, tablet, capsule, liquid, or sustained release tablet for oral administration; or a liquid for intravenous, subcutaneous, or parenteral administration; or a polymer or other sustained release vehicle for local administration.

Methods well known in the art for making formulations are found, for example, in “Remington: The Science and Practice of Pharmacy” (20th ed., ed. A. R. Gennaro, 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids and the like; polymeric acids such as tannic acid, carboxymethyl cellulose, and the like; and inorganic acid such as hydrochloric acid, hydrobromic acid, sulfuric acid phosphoric acid, and the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depend on various clinical factors including the overall health of the subject and the severity of the symptoms of disease, e.g., cancer. In general, once a tumor is detected, administration of the agent is used to treat or prevent further progression of the tumor. Treatment can be performed for a period of time ranging from 1 to 100 days, more preferably 1 to 60 days, and most preferably 1 to 20 days, or until the remission of the tumor. It is understood that many chemotherapeutic agents are not administered daily, particularly agents with a long half-life. Therefore, an agent can be continually present without being administered daily. Dosages vary depending on each compound and the severity of the condition. Dosages can be titrated to achieve a steady-state blood serum concentration. Dosages can be interrupted or decreased in the presence of dose limiting toxicities.

### III. METHODS OF THE INVENTION

The instant invention provides methods of identifying a subject who will likely respond favorably to treatment with an Hsp90 inhibitor by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, or fecal matter, for the presence of one or more indicators of the level of hypoxia in the tumor.

The specific subject sample analyzed will depend, for example, on the site of the tumor. It is known that hypoxia drives angiogenesis in tumors, resulting in leaky blood vessels resulting in the presence of markers in circulation. Further, tumor growth and hypoxia are typically associated with necrosis and cell breakdown, resulting in cellular material in other bodily fluids or wastes. These readily accessible subject samples allow for the monitoring of the subject for the presence, or absence, of markers for hypoxia prior to and during the course of treatment.

Biopsies are routinely obtained for the purpose of cancer diagnosis, and solid tumors are frequently further resected prior to initiation of chemotherapy which also can be used for analysis to determine the level of hypoxia. Biopsy samples and resected tumor samples typically include at least some normal tissue adjacent to the tumor that can be used as a control.

In one embodiment of the invention, the modulated level of hypoxia is a high level of hypoxia. In one embodiment of the invention, the modulated level of hypoxia is a high level of LDH.

In one embodiment, the level of hypoxia is determined by detecting the level of one or more hypoxia-modulated polypeptides or using one or more methods such as imaging methods. In one embodiment, a hypoxia-modulated polypeptide is at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR), neurolipin 1 (NRP-1), pyruvate dehydrokinase (PDH-K), and omithine decarboxylase (ODC). In one embodiment, the isoform or subunit of LDH is LDHH, LDH5, LDH4, LDH3, LDH2, LDH1 or LDHM, or any combination thereof. In another embodiment, the isoform or subunit of LDH is LDH5. In another embodiment, the level of hypoxia is determined by determining the ratio of two or more forms of LDH, e.g., the ratio of LDH5:LDH1. In another embodiment, the isoform of HIF is HIF-1α, HIF-1β, HIF-2α, and HIF-2β. In another embodiment, the pro-angiogenic isoform of VEGF is any one or a combination of VEGF-A splice variants. Antibodies against prodrugs that localize in hypoxic regions (e.g., EF5, pimonidazole, etc.) can also be used to detect hypoxia. Tumor size can also be correlated with a level of hypoxia. A level of hypoxia can also be determined by PET scan. Functional imaging measuring blood flow in the tumor can be used as an indicator of hypoxia in the tissue. Direct measurement of hypoxia can be performed by inserting a sensor into the tumor.

Methods to detect the protein or activity levels of markers of hypoxia, or hypoxia-modulated polypeptides, are well known in the art. Antibodies against and kits for detection of hypoxia-modulated polypeptides can be purchased from a number of commercial sources. Alternatively, using routine methods known in the art (e.g., immunization of animals, phage display, etc.) antibodies against one or more hypoxia-modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Alternatively, immunohistochemical methods can be used on tumor samples and tissue sections. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample. Many markers of hypoxia, including LDH, are enzymes. Enzymatic activity can be assayed in total, or for individual isoforms, for example, using in gel assays.

Alternatively, nucleic acid based methods of detection of levels of hypoxia are also well known in the art. Methods of designing primers and probes for quantitative reverse transcription real time (rt) PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art.

In another aspect, the present invention provides methods for the pre-selection of a subject for therapeutic treatment with an anti-cancer agent, wherein the subject has previously been found to have a high level of hypoxia. The invention also provides methods for the pre-selection of a subject for therapeutic treatment with an agent by evaluating the results of an assessment of a sample from the subject for a high level of hypoxia.

Such determinations can be made based on a chart review of the level of hypoxia of the tumor of the subject. Inclusion criteria can include information being available regarding the cancer type, the specific treatment regimen with the agent, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information can be considered when available. Exclusion criteria can include the presence of other diseases or conditions that could result in alteration of levels of hypoxia-modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The subjects can be sorted into groups based on various criteria. Subjects who were treated with an agent for whom no levels of hypoxic markers were determined can be used as an unstratified control group to understand the efficacy of the agent on a treatment population not selected based on the level of hypoxia in the subject. Alternatively, the population analyzed in the study can be compared to historical control samples in which an unstratified population was analyzed for response to the agent.

Subjects for whom hypoxic levels were obtained can be divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with the agent, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; GLUT-1, GLUT-2, neurolipin 1 (NRP-1), pyruvate dehydrokinase (PDH-K), and ornithine decarboxylase (ODC). Tumor size can also be a marker correlated with a level of hypoxia. A marker of a level of hypoxia can also be determined by PET scan. A level of hypoxia can also be determined by PET scan. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. Functional imaging measuring blood flow in the tumor can be used as an indicator of hypoxia in the tissue. Direct measurement of hypoxia can be performed by inserting a sensor into the tumor. Antibodies against prodrugs that localize in hypoxic regions (e.g., EF5, pimonidazole, etc.) can also be used as markers to detect hypoxia. Functional imaging measuring blood flow in the tumor can be used as a marker of hypoxia in the tissue. Direct measurement of hypoxia can be performed to provide a marker for hypoxia by inserting a sensor into the tumor. Again, it is preferred that the same method of determining the level of the marker of hypoxia is used for all samples, particularly when qualitative assessment methods are used.

Outcomes of subjects based on the level of hypoxia can be analyzed to determine if the outcome between the two groups is different. Outcomes can further be compared to a non-stratified group treated with the Hsp90 inhibitor. Methods for statistical analysis and determination of statistical significance are within the ability of those of skill in the art. The analysis demonstrates that subjects with a high level of hypoxia have a better response, e.g., one or more of longer time to failure, longer survival time, better quality of life, decreased tumor size, better tolerance of the agent, etc., as compared to subjects with a low level of hypoxia.

In another aspect, the present invention provides methods for the pre-selection of a subject for therapeutic treatment with an Hsp90 inhibitor, wherein the subject has previously been found to have a high level of hypoxia. The invention also provides methods for the pre-selection of a subject for therapeutic treatment with an Hsp90 inhibitor by evaluating the results of an assessment of a sample from the subject for a modulated level of hypoxia wherein the subject is found to have a high level of hypoxia. Such determinations can be made based on the level of hypoxia observed in historical samples. An analysis using samples collected from subjects during treatment can be performed to determine the efficacy of a selected agent for the treatment of cancer based on the level of hypoxia of the tumor based on markers assessed during the treatment of the subjects. Inclusion criteria are information being available regarding the cancer type, the specific treatment regimen with the selected agent, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information is considered when available. Exclusion criteria can include the presence of other diseases or conditions that could result in alteration of levels of hypoxia-modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The samples can be analyzed for the level of hypoxia. Preferably, all of the samples are the same type or types, e.g., blood, plasma, lymph, or tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia can be measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, GLUT-1, GLUT-2, neurolipin 1 (NRP-1), pyruvate dehydrokinase (PDH-K), and omithine decarboxylase (ODC). Enzymatic assays of markers can be performed. Tumor size can also be a marker correlated with a level of hypoxia. A marker of a level of hypoxia can also be determined by PET scan. Antibodies against prodrugs that localize in hypoxic regions (e.g., EF5, pimonidazole, etc.) can also be used as markers to detect hypoxia. Functional imaging measuring blood flow in the tumor can be used as a marker of hypoxia in the tissue. Direct measurement of hypoxia can be performed to provide a marker for hypoxia by inserting a sensor into the tumor. Further, it is preferred that the same type of subject sample, e.g., blood, serum, lymph, tumor tissue, etc., is tested for the presence of the marker for the level of hypoxia. It is understood that the level of hypoxia could have been measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Again, it is preferred that the same method of determining the level of the marker of hypoxia is used for all samples, particularly when qualitative assessment methods are used.

In another aspect, the present invention provides methods for treating a cancer with an Hsp90 inhibitor in a subject having a high level of hypoxia. The methods include not administering to the subject having a cancer or susceptible to a cancer who further has a low level of hypoxia, an Hsp90 inhibitor, thereby treating the cancer. Other methods include administering to the subject having a cancer or susceptible to a cancer an Hsp90 inhibitor, and at least one chemotherapeutic agent, thereby treating the cancer. In certain embodiments, the subject has previously been treated with a chemotherapeutic agent.

Other methods include methods of treating a subject who has cancer by prescribing to the subject an effective amount of an Hsp90 inhibitor, wherein the subject has previously been found to have a high level of hypoxia. As used herein, the term “prescribing” is understood as indicating a specific agent or agents for administration to a subject. Furthermore, the present invention also includes methods of increasing the likelihood of effectively treating a subject having cancer by administering a therapeutically effective amount of a composition comprising an Hsp90 inhibitor, to the subject, wherein the subject has previously been found to have a modulated level of hypoxia.

Cancers that may be treated or prevented using the methods of the invention include, for example, acoustic neuroma, acute leukemia, acute lymphocytic leukemia, acute myelocytic leukemia (monocytic, myeloblastic, adeno carcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. Other cancers include primary cancer, metastatic cancer, oropharyngeal cancer, hypopharyngeal cancer, liver cancer, gallbladder cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, and pituitary gland cancer, hemangiomas, sarcomas arising from bone and soft tissues; Kaposi's sarcoma, nerve cancer, ocular cancer, and meningial cancer, glioblastomas, neuromas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, squamous cell carcinoma of the head and neck (SCCHN), nasopharageal cancer, oral cancer, biliary tract, hepatocellular carcinoma, non-medullary thyroid carcinoma, recurrent glioblastoma multiforme, neurofibromatosis type 1, CNS cancer, liposarcoma; leiomyosarcoma; salivary gland cancer, mucosal melanoma; acral/lentiginous melanoma, paraganglioma, and pheochromocytoma.

It is understood that diagnosis and treatment of a complex disease such as cancer is not performed by a single individual, test, agent, or intervention. For example, a subject may meet with a primary care physician to express a concern and be referred to an oncologist who will request tests that are designed, carried out, and analyzed by any of a number of individuals, but not limited to, radiologists, radiology technicians, physicists, phlebotomists, pathologists, laboratory technicians, and radiation, clinical, and surgical oncologists. Selection, dosing, and administration of agents to a subject diagnosed with cancer will be performed by any of a number of individuals including, but not limited to, radiologists, radiology technicians, physicists, pathologists, infusion nurses, pharmacists, and radiation, clinical, and surgical oncologists. Therefore, it is understood that within the terms of the invention, identifying a subject as having a specific level of hypoxia can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject, e.g., by an identification card, hospital bracelet, asking the subject for his/her name and/or other personal information to confirm the subjects identity.

Similarly, administering an Hsp90 inhibitor can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

As discussed extensively, above, the terms “administer”, “administering” or “administration” include any method of delivery of a pharmaceutical composition or agent into a subject's system or to a particular region in or on a subject. In certain embodiments of the invention, an Hsp90 inhibitor is administered intravenously, intramuscularly, subcutaneously, intradermally, intranasally, orally, transcutaneously, or mucosally. In a preferred embodiment, an agent is administered intravenously. Administering an Hsp90 inhibitor can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

In certain embodiments, the invention further provides a business method for decreasing healthcare costs comprising:
determining the level of hypoxia in a biological sample from a cancer obtained from a subject;
storing the information on a computer processor;
determining if the subject would likely benefit from treatment with an Hsp90 inhibitor based on the level of hypoxia; and
treating the subject only if the subject will likely benefit from treatment,
thereby decreasing healthcare costs.

In certain embodiments of the business method, the subject has a solid tumor. In certain embodiments of the business method, the subject has primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, or refractory malignancy.

In certain embodiments of the business method, the level of hypoxia in a tumor is determined in a subject sample. In certain embodiments, the subject sample may be tumor tissue, blood, urine, lymph, cerebrospinal fluid, circulating tumor cells, bronchial lavage, peritoneal lavage, exudate, effusion, or sputum. In certain embodiments, the tumor tissue is in the subject. In certain embodiments, the tumor tissue is removed from the subject.

In certain embodiments of the business method, the level of hypoxia is determined by detecting the level of one or more hypoxia-modulated polypeptides. In certain embodiments, the hypoxia-modulated polypeptides are up regulated in the sample. In certain embodiments, the level of hypoxia is determined by detecting the activity level or expression level of one or more hypoxia-modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments of the business method, the isoform or subunit of LDH comprises one or more of LDH5, LDH4, LDH3, LDH2, LDH1, LDHA and LDHB; or any combination thereof including total LDH. In certain embodiments, the isoform of HIF may be HIF-1α, HIF-1β, HIF-2α, or HIF-2β; or any combination thereof including total HIF-1 and HIF-2. In certain embodiments, the pro-angiogenic isoform of VEGF is VEGF-A, or any combination thereof including total VEGF-A.

In certain embodiments of the business method, the detection of a high level of activity or expression of at least one LDH isoform or subunit comprises detection of an LDH activity or expression level of an LDH selected from the group consisting of total LDH, LDH5, LDH4; LDH5 plus LDH4; LDH5 plus LDH4 plus LDH3; and LDHA, wherein the activity level or expression level is 0.8 ULN or more.

In certain embodiments of the business method, the detection of a high level of activity or expression of at least one LDH isoform or subunit comprises detection of an LDH activity or expression level of an LDH selected from the group consisting of total LDH, LDH5, LDH4; LDH5 plus LDH4; LDH5 plus LDH4 plus LDH3; and LDHA, wherein the activity level or expression level is 1.0 ULN or more.

In certain embodiments of the business method, the high level of hypoxia is a change in a ratio or a ratio of normalized levels of hypoxia-modulated polypeptides. In certain embodiments, the high level of hypoxia comprises a ratio or a normalized ratio of 1.0 or more of the ULN, wherein the ratio or normalized ratio may be LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, or LDH3 to total LDH.

In certain embodiments of the business method, the subject was previously treated with another chemotherapeutic agent.

In certain embodiments of the business method, the Hsp90 inhibitor may be ganetespib, geldanamycin (tanespimycin), IPI-493, macbecins, tripterins, tanespimycins, 17-AAG (alvespimycin), KF-SS823, radicicols, KF-S8333, KF-S8332, 17-DMAG, IPI-S04, BIIB-021, BBB-028, PU-H64, PU-H71, PU-DZ8, PU-HZ1S1, SNX-2112, SNX-2321, SNX-S422, SNX-7081, SNX-8891, SNX-0723, SAR-S67S30, ABI-287, ABI-328, AT-13387, NSC-113497, PF-3823863, PF-4470296, EC-102, EC-154, ARQ-250-RP, BC-274, VER-50589, KW-2478, BHr-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i), herbinmycin A, radicicol, CCT018059, PU-H71, or celastrol. In certain embodiments of the business method, the Hsp90 inhibitor is not ganetespib.

### IV. KITS OF THE INVENTION

The invention also provides for kits to practice the methods of the invention. For example, a kit can include an Hsp90 inhibitor, and an instruction for administration of the selected agent to a subject having cancer with a high level of hypoxia. In another embodiment, the subject has cancer with a high level of lactate dehydrogenase (LDH). In one embodiment, the instruction provides that the Hsp90 inhibitor is a second line therapy. In another embodiment, the kits of the invention may contain reagents for determining the level of LDH in a sample from a subject.

### EXAMPLES

### Example 1

### A Phase I Dose Escalation Study of Ganetespib in Twice-Weekly Administration in Patients with Solid Tumors

An open-label Phase 1 dose-escalation study in subjects with solid tumors was performed. The first cohort consisted of three subjects who received 2 mg/m² of ganetespib during a 1-hour infusion 2 times per week (e.g., [Monday, Thursday] or [Tuesday, Friday]) for three consecutive weeks followed by a 1 week dose-free interval. The first infusion for the first three subjects was staggered by a minimum of 5 days between subjects. This staggered enrollment scheme was followed for the first cohort only. Subjects tolerating ganetespib continued treatment past week 8 until disease progression as long as the re-treatment criteria continued to be met.

Subsequent cohorts were originally planned to receive escalating doses of 4, 7, 10, 14, 19, 25, 33, 40, and 48 mg/m², provided that the previous dose was well-tolerated during cycle 1 (week 1-4). The dose escalation scheme was updated to 25, 50, 75 and 100 mg/m², provided that the previous dose was well-tolerated during cycle 1. Following the completion of enrollment at 50 mg/m² twice per week, subsequent cohorts were to be treated at 100 mg/m² (100% increase above prior dose) and 120 mg/m² (20% increase above prior dose), with further dose increments to be approximately 20% over the previous dose level, until the maximum tolerated dose (MTD) was determined. Enrollment was completed at 100 mg/m² and the next doses planned were 120 mg/m² and 144 mg/m².

There had to be at least three evaluable subjects in a cohort before dose escalation could occur. An evaluable subject was defined as one who had received at least 5 of 6 doses of ganetespib during cycle 1 and had a subsequent follow up visit or experienced a dose limiting toxicity (DLT) after any dose. Once a subject experienced a DLT the cohort was expanded to six subjects. If only 1 of 6 subjects experienced a DLT, further dose escalation was allowed. However, if 2 of 3 or 2 of 6 subjects experienced a DLT, dose escalation terminated.

A subject's duration of participation included a 2-week screening period and two 4-week treatment cycles totaling approximately 10 weeks. However, at the investigator's discretion, subjects tolerating ganetespib continued treatment past week 8 until disease progression.

The subjects in this study had histologically- or cytologically-confirmed non-hematological malignancy that was metastatic or unresectable. The subjects were documented to be refractory to, or were not candidates for, current standard therapy.

Ganetespib was formulated using 90% v/v PEG 300 and 10% v/v Polysorbate 80 at a concentration of 8 mg/mL and was packaged in a Type I glass amber vial, stoppered with a Flurotec-coated stopper, and sealed. Each vial had a deliverable volume of 12.5 mL (equivalent to 100 mg/vial). The formulation was further diluted with 5% dextrose for injection in infusion container (DEHP-free 500 mL) to a concentration range of 0.02 to 1.2 mg/mL and administered via infusion tubing (DEHP-free) with a 0.22 micron end filter over an hour to the patient. The dosing solution once prepared was administered within 3 hours. Eligible subjects received the drug during a 1-hour infusion 2 times per week for 3 consecutive weeks followed by a 1-week dose-free interval. The amount of ganetespib administered depended upon the cohort to which the subject was assigned and the subject's body surface area (BSA). This cycle was repeated for subjects tolerating ganetespib who did not experience disease progression.

Forty-one of 54 enrolled patients were assessable for response. A total of 13 patients discontinued prior to the Week 8 response assessment. Confirmed partial responses included 1 patient with melanoma and 1 patient with triple negative breast cancer. Fifteen (15) patients achieved stable disease.

### Example 2

### Efficacy of Ganetespib in the Treatment of Triple Negative Breast Cancer Subject from Example 1

Triple-negative breast cancer (TNBC) represents 10-20% of all diagnosed breast cancer cases and tests negative for the presence of estrogen receptor (ER), progesterone receptor (PR), and the human epidermal growth factor receptor 2 (HER2). Therefore, this breast cancer subtype does not respond to hormonal therapy used to treat breast cancer, such as tamoxifen or aromatase inhibitors, or therapies that target HER2 receptors, such as Herceptin®. Triple-negative breast cancer is characterized as more aggressive than other breast cancer subtypes, disproportionately affects younger women, and is associated with a poorer 5-year survival rate of 77%, as compared to the 93% survival rate for other cancers. Triple-negative breast cancer is typically treated with a combination of therapies such as surgery, radiation therapy, and chemotherapy, however, early relapse and metastasis is common.

A 39-year old white female with triple negative breast cancer (with Stage III invasive ductal carcinoma) from Example 1 enrolled in a Phase I dose escalation study of ganetespib. The patient's disease had progressed on 7 prior chemotherapeutic regimens. The patient was administered 144 mg/m² of ganetespib twice-weekly for 3 weeks, followed by 1 week dose-free. After 2 cycles, she demonstrated stable disease per Response Evaluation Criteria in Solid Tumors (RECIST). After 4 cycles, there was a documented 31% reduction in target lesion size (partial response). The treatment was interrupted due to brain metastases treated with whole brain radiation, but treatment with ganetespib resumed in cycle 5. The patient tolerated the treatment well with mild/moderate toxicities.

### Example 3

### Efficacy of Ganetespib in the Treatment of Non-Small Cell Lung Cancer (NSCLC) by KRAS, EGFR, and ALK Mutation Status

A Phase 2 clinical trial was performed to determine the efficacy of ganetespib in the treatment of NSCLC.

Patients with advanced NSCLC who failed prior treatments received 200 mg/m² of ganetespib as a 1-hr infusion once weekly for 3 of a 4-wk cycle in a Simon two-stage study design assessing primary endpoint of PFS rate at 16 wks. Initial cohorts were defined by mutation status: A) EGFR mutation, KRAS wild-type B) EGFR wild-type, KRAS mutation C) EGFR wild-type, KRAS wild-type (WT). If ≧2/14 patients in A, B or C were progression-free at 16 weeks, enrollment increased to 23 patients for that cohort. Tumor response was assessed every 8 weeks. Cohort D was added to include 35 additional EGFR wild-type and KRAS wild-type patients with adenocarcinoma histology. FISH analysis for ALK translocation, was performed for Cohorts C and D.

There were 73 patients (31 M, 42 F; median age 62 years, range 28-82; ECOG 0-1; prior therapies range 1-10) that received a median of 2 cycles (range 1-12) of ganetespib in cohorts A (14), B (17), and C+D (42). Adverse events reported in ≧20% of patients included diarrhea, fatigue, nausea, anorexia, constipation, and dyspnea and were generally grade 1-2.

In Cohort B, subjects with a wild-type EGFR and a KRAS mutation, greater than 60% of patients with NSCLC exhibited tumor shrinkage at 8 weeks, indicating that ganetespib is useful in the treatment of NSCLC with a KRAS mutation.

Expansion criteria were achieved for cohort C, including a durable partial response (PR) and seven patients with prolonged stable disease (≧16 weeks).

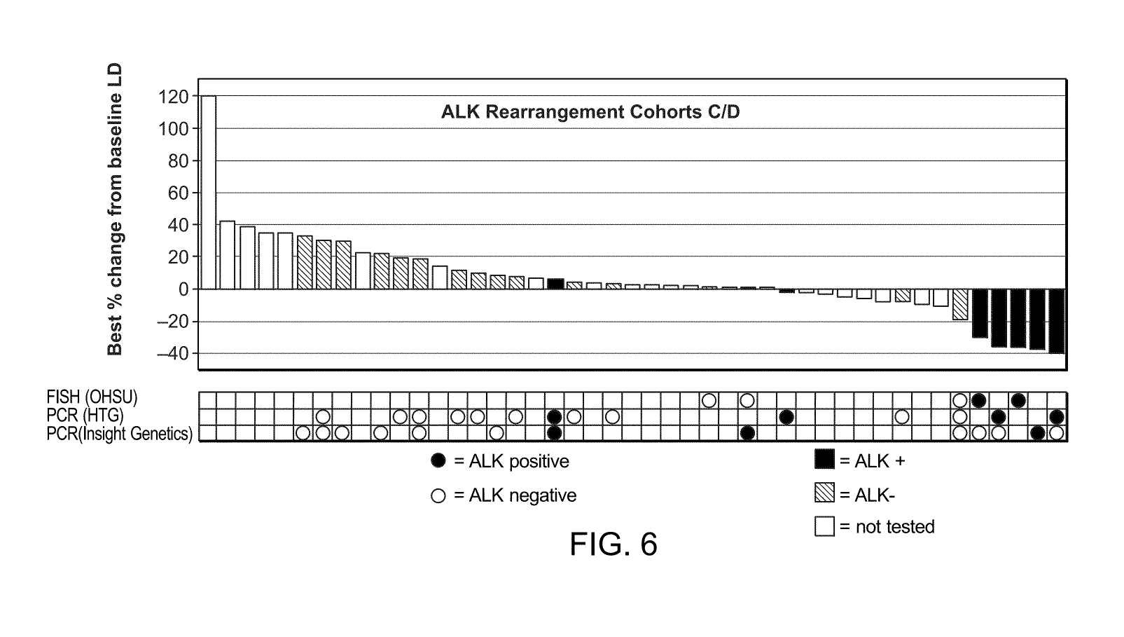

Of the 23 patients of cohorts C and D (EGFR wild-type, KRAS wild-type) in the Phase 2 trial tested for ALK translocation or rearrangement (ALK+), eight patients were ALK+ in at least one assay. Six of these eight ALK+ patients (75%) showed tumor shrinkage in target lesions. One ALK+ patient showed no change in tumor size, and one ALK+ patient achieved stable disease (tumor growth <20%). The disease control rate in this population was 7/8 (88%), and the objective response rate (complete response (CR)+partial response (PR)) was 4/8 (50%) (See FIG. 6).

In summary, ganetespib administered as a single-agent was well-tolerated in patients with NSCLC at 200 mg/m² once weekly without severe liver, ocular, cardiovascular or renal toxicity. Clinical activity was observed in patients with advanced NSCLC tumors with both a wild-type EGFR and a KRAS mutation; a wild-type EGFR and a wild-type KRAS. Clinical activity was observed in patients with ALK+ NSCLC tumors (i.e., tumors with an ALK mutation). This demonstrates the utility of ganetespib for the treatment of NSCLC with various mutations.

### Example 4

### Efficacy of Ganetespib in a Phase 2 Study for the Treatment of Gastrointestinal Stromal Tumors (GIST)

A gastrointestinal stromal tumor (GIST) is a type of cancer that occurs in the gastrointestinal (GI or digestive) tract, including the esophagus, stomach, gall bladder, liver, small intestine, colon, and rectum. The American Cancer Society estimates 4,500 to 6,000 GIST cases are diagnosed each year in the United States. Although these tumors can start anywhere in the GI tract, they occur most often in the stomach (50% to 70%) or the small intestine (20% to 30%). Gastric cancer is second to lung cancer as the most lethal cancer worldwide, with 5-year survival rates in the range of 10% to 15%.

Patients with advanced (e.g., metastatic or unresectable) GIST following failure of prior therapy, e.g., imatinib or sunitinib, received ganetespib (200 mg/m²) as a 1 hour IV infusion once per week for 3 weeks of a 28 day cycle. GIST status was assessed at 8 weeks per RECIST, until progression. In this Simon's 2 stage study design, if ≧4/23 patients in Stage 1 had clinical benefit (CR+PR+stable disease (SD)≧16 weeks) enrolment would continue with Stage 2. Hsp90 client protein levels were analyzed in biopsies pre-therapy and 24-48 h post-treatment with ganetespib in a subset of patients.

There were 26 patients (15 M, 11 F; median age 53 years, range 33-67; ECOG status 0-1; median 5 prior therapy regimens, range 3-12, wild-type platelet derived growth factor receptor alpha (PDGFRA)) that received a median of 2 cycles of ganetespib (range 1-8). Adverse events reported in >20% of patients were generally NCI CTC grade 1-2 and included diarrhea, fatigue, nausea, vomiting, increased alkaline phosphatase, headache, insomnia, and abdominal pain. At the time of the analysis, 23 patients out of 26 patients in the intent to treat (ITT) population were evaluable. 12/23 evaluable patients had SD (4 SD 16 weeks, 8 SD≧8 weeks), meeting formal criteria to enroll Stage 2. However, analysis of client proteins in paired tumor biopsies from 4 patients did not show prolonged inhibition of activated KIT or its downstream pathways.

In summary, ganetespib given by once-weekly dosing was well-tolerated in patients with heavily pre-treated advanced GIST, with no evidence of severe liver, ocular, cardiac or renal toxicity. Disease stabilization was seen in a subset of patients. These results demonstrate the utility of ganetespib in the treatment of GIST.

### Example 5

### Efficacy of Ganetespib in a Phase 2 Study for the Treatment of Solid Tumors

A phase 2 study of ganetespib was performed to determine its efficacy in the treatment of solid tumors.

Patients with solid tumors who had exhausted standard treatment options received ganetespib as a 1 hr infusion twice-weekly for 3 weeks (weeks) of a 28 day cycle until disease progression. Serial PK and pharmacodynamic samples were obtained during cycle 1. Safety assessments included frequency and grade of adverse events (AEs), laboratory parameters and ECG changes.

Data were presented for 49 patients (22 M, 27 F; median age 55 years, range 32-81; ECOG status range 0-2) treated at doses from 2-144 mg/m². Patients received a median of 2 (range 1-12) cycles of ganetespib. AEs reported in ≧20% of patients treated at doses from 2-120 mg/m² were fatigue, diarrhea, nausea, anemia, abdominal pain, constipation, anorexia, vomiting, and headache; the majority of events were mild to moderate in severity with absence of severe liver, ocular, cardiac and renal toxicity. Two DLTs (elevated transaminases) were reported in the 10 and 144 mg/m² cohorts. Ganetespib showed linear PK, rapid distribution, a mean terminal half-life of 10-14 hours, a volume of distribution greater than total body water and no accumulation in plasma. A confirmed durable PR by RECIST was seen in a patient with metastatic melanoma. Additionally, 2 NSCLC patients who received 6 months of treatment had durable SD, with tumor shrinkage.

In summary, ganetespib was well-tolerated administered twice-weekly. Preliminary safety profile, activity signals and differences in client protein kinetics warrant continued evaluation of ganetespib using a twice-weekly dosing regimen.

### Example 6

### A Phase 2 Trial of Ganetespib: Efficacy and Safety in Patients with Metastatic Breast Cancer (MBC)

A phase 2 trial was performed to determine the safety and efficacy of ganetespib in the treatment of subjects with metastatic breast cancer.

Patients with locally advanced or MBC were treated with single agent of ganetespib at 200 mg/m² on a cycle of once weekly for 3 weeks, one week off, on a 28 day cycle. The primary endpoint of the trial was overall response rate using RECIST 1.1. Patients with HER2+ breast cancer were required to have received prior therapy with trastuzumab. No more than 3 lines of chemotherapy in the metastatic setting were permitted, but there was no limit on prior lines of hormone therapy. Patients were evaluated for response after 2 cycles. The trial used a Simon two-stage design requiring at least 3 responses among the first 22 patients, to allow expansion to a total of 40 patients.

A total of 22 patients were treated with a median age of 51 years (38 to 70) and the following subtypes: 13 HER2+ (10 ER+/HER2+; 3 ER−/HER2+), 6 ER+/HER2−, and 3 ER−/PR−/HER2− (TNBC).

Prior treatment regimens are summarized as follows:

| | Number of Subjects |
|---|---|
| Prior lines of chemotherapy | |
| in metastatic setting | |
| 1 | 8 |
| 2 | 9 |
| 3 | 5 |
| Prior lines of trastuzumab | |
| in metastatic setting | |
| 0 | 9 |
| 1 | 6 |
| 2 | 6 |
| 3 | 1 |

The responses of the subjects by ER, PR, and HER2 status are summarized in the table below.

| | Total | ER+/HER2− | HER2+ | TNBC |
|---|---|---|---|---|
| Response | (N = 22)* | (N = 6) | (N = 13)* | (N = 3) |
| ORR | 2 (9%) | 0 | 2 (9%) | 0 |
| CR | 0 | 0 | 0 | 0 |
| PR | 2 (9%) | 0 | 2 | 0 |
| SD | 7 (32%) | 0 | 6 (27%) | 1 (5%) |
| CBR* | 2 (9%) | 0 | 2 (9%) | 0 |

*(CR + PR + SD > 6 months)

These were the first data showing an objective anti-tumor response with single agent Hsp90 inhibitor therapy in patients with advanced breast cancer. Additionally, these were the first data to show anti-tumor activity for an Hsp90 inhibitor in TNBC. In this study, the single agent of ganetespib was well tolerated, with expected GI toxicity that was mild in nature and manageable in all patients.

### Example 7

### Ganetespib Displays Activity Across Breast Cancer Subtypes

Breast cancer is a heterogeneous disease historically broken down into 4 subtypes. Various compounds were tested for their effects in cell viability assays using various breast cancer cell lines. Cellular viability was assessed using the CellTiter-Glo® Luminescent Cell Viability Assay (Promega, Madison, Wis., USA) according to the manufacturer's protocol. KRAS mutant NSCLC cell lines were seeded into 96-well plates based on optimal growth rates determined empirically for each line. Twenty-four hours after plating, cells were dosed with graded concentrations of ganetespib for 72 h. CellTiter-Glo® was added (50% v/v) to the cells, and the plates incubated for 10 min prior to luminescent detection in a SpectraMax® Plus 384 microplate reader (Molecular Devices, Sunnyvale, Calif., USA). Data were normalized to percent of control and IC50 values used to determine the sensitivity of each line. For the comparative analysis study with MEK and PI3K/mTOR inhibitors, A549, H2009, Calu-1, and H358 cells were treated with graded concentrations of ganetespib, AZD6244, or BEZ235 for 72 h and cell viability measured as above.

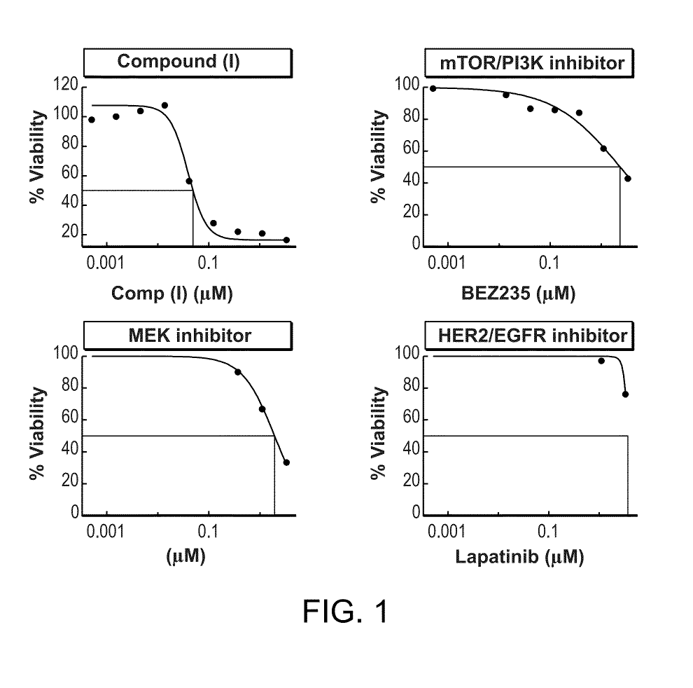

Shown in FIG. 1, ganetespib showed potency across all 4 subtypes (luminal HER2+, luminal HER2−, Basal A, Basal B) of breast cancer cells, grown as a monolayer in vitro. The IC50s of the various compounds and the ER, PR, and HER2 status are provided in the table below.

Breast cancer, cell lines, marker status, and IC50 in response to ganetespib at 72 hr (viability by CTG).

| Cell Line | Subtype | ER | PR | HER2 | IC50, nM |
|---|---|---|---|---|---|
| OCUB-M | Basal | −− | −− | | 39 |
| MDA-MB-468 | Basal A | −− | −− | | 27 |
| HCC70 | Basal A | −− | −− | | 114 |
| MDA-MB-231 | Basal B | −− | −− | | 24 |
| SK-BR-3 | Luminal | −− | −− | + | 10 |
| BT-474 | Luminal | + | + | + | 13 |
| MCF7 | Luminal | + | + | | 25 |

Basal breast cancer is a subtype believed to be more stem like and less differentiated than luminal breast cancer, and therefore more aggressive with limited treatment options. Comparison was made for the anticancer activity of ganetespib versus MEK and mTOR inhibitors in the basal line MDA-MB-231, using lapatinib as a control since these cells were HER2 negative. Shown in FIG. 1, ganetespib was highly potent, killing all the cells as opposed to the weak activity of the mTOR and MEK inhibitors.

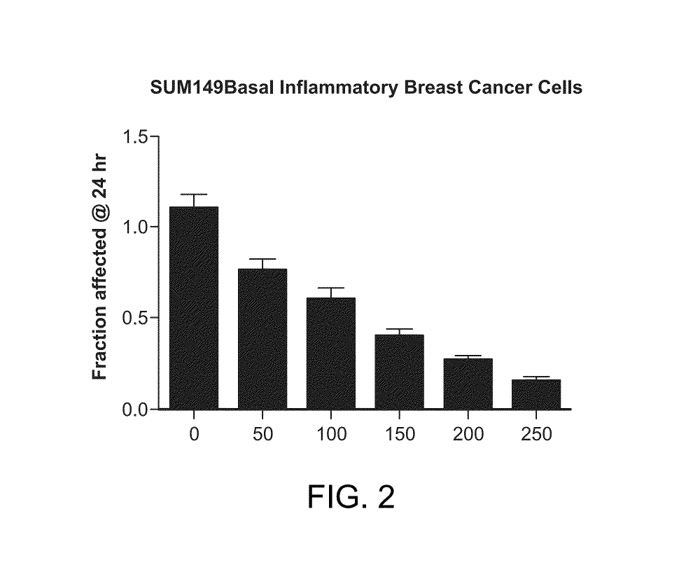

Ganetespib was assayed in inflammatory breast cancer (IBC), a rare but aggressive form of breast cancer distinct from the subtypes presented above. Shown in FIG. 2, ganetespib displayed considerable anticancer activity against SUM149 cells 24 hr after exposure.

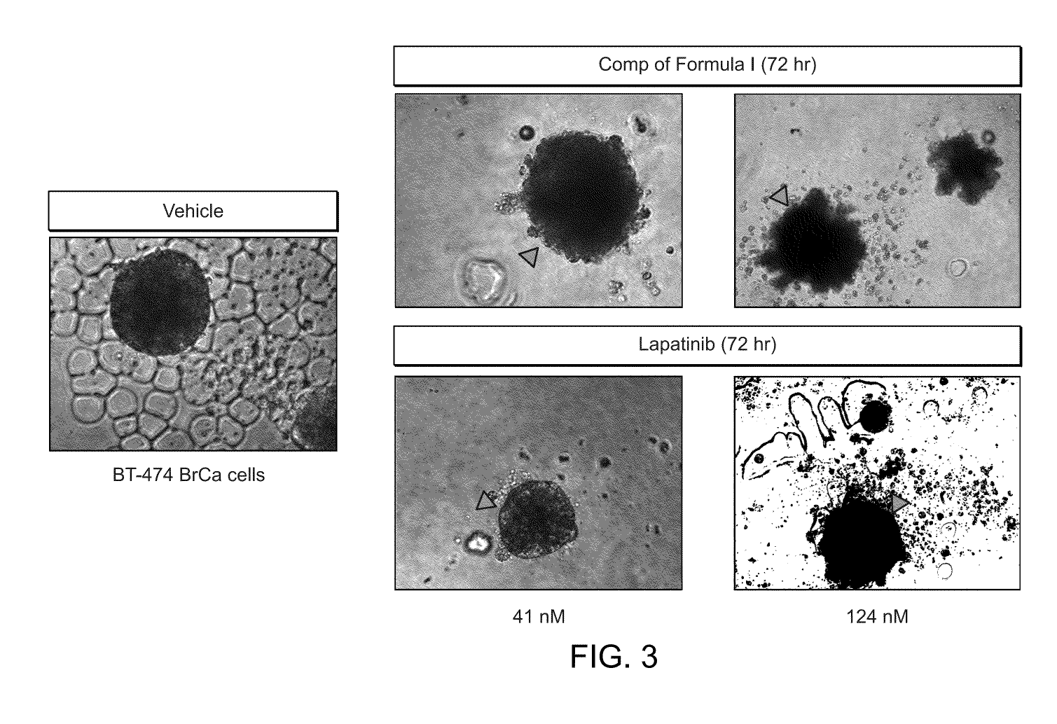

BT-474 HER2+ luminal cells were cultured as mammospheres in Matrigel® and exposed to ganetespib for 72 hr. As shown in FIG. 3, ganetespib was fully capable of killing cells organized into spheroids, with an IC50 (20 nM) nearly identical to that observed in 2D (13 nM), demonstrating that ganetespib retained its activity in breast cancer cells grown in three dimensions.

### Example 8

### Expression of Hsp90 Client Proteins in BT-474 HER2+Luminal Breast Cancer Cells after Treatment with Ganetespib

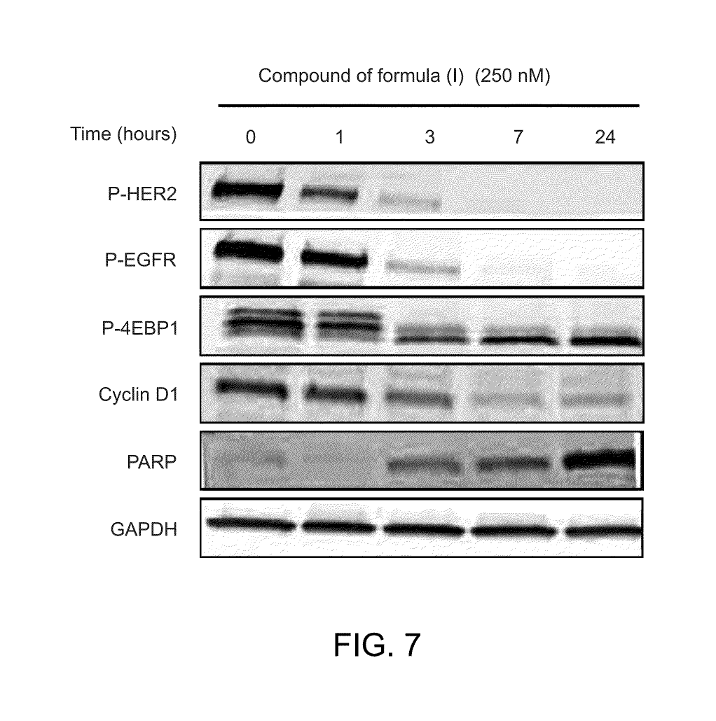

Expression of various proteins in the BT-474 HER2+ luminal breast cancer cells after exposure to ganetespib was assessed by western blot using routine methods. Briefly, following treatment, tumor cells were disrupted in lysis buffer (CST) on ice for 10 min. Lysates were clarified by centrifugation and equal amounts of proteins resolved by SDS-PAGE before transfer to nitrocellulose membranes (Invitrogen, Carlsbad Calif.). Membranes were blocked with 5% skim milk in TBS with 0.5% Tween and immunoblotted with the indicated antibodies. Antibody-antigen complexes were visualized using an Odyssey system (LI-COR, Lincoln, Nebr.). FIG. 7 is a western blot showing expression of various proteins in the BT-474 HER2+ cells at various time points after treatment with ganetespib.

### Example 9

### Treatment of Breast Cancer with Ganetespib and BEZ235 in a Mouse Xenograft Tumor Model

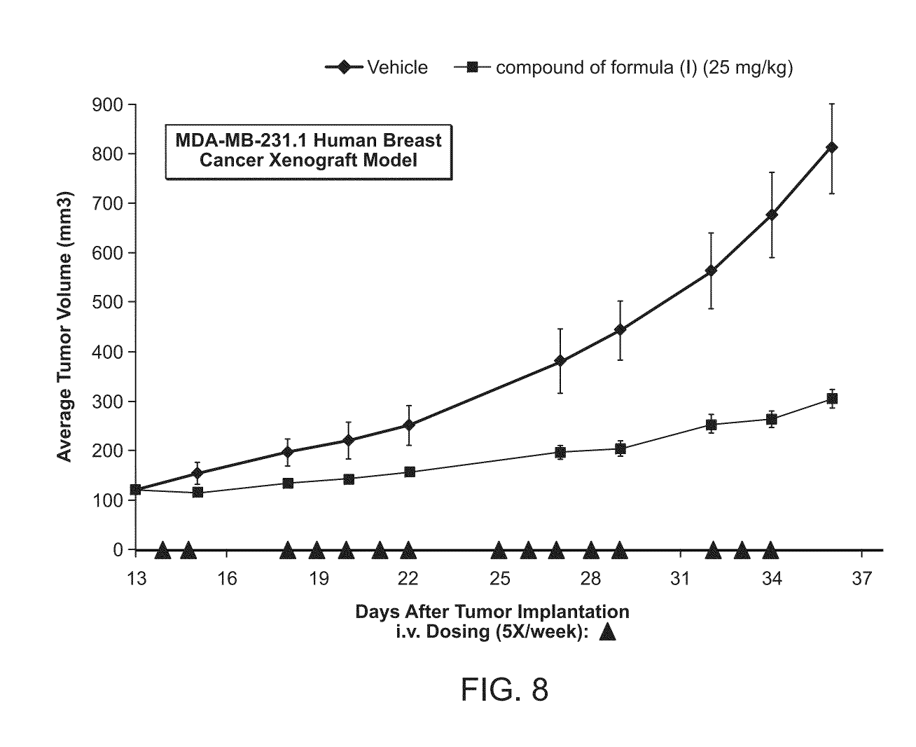

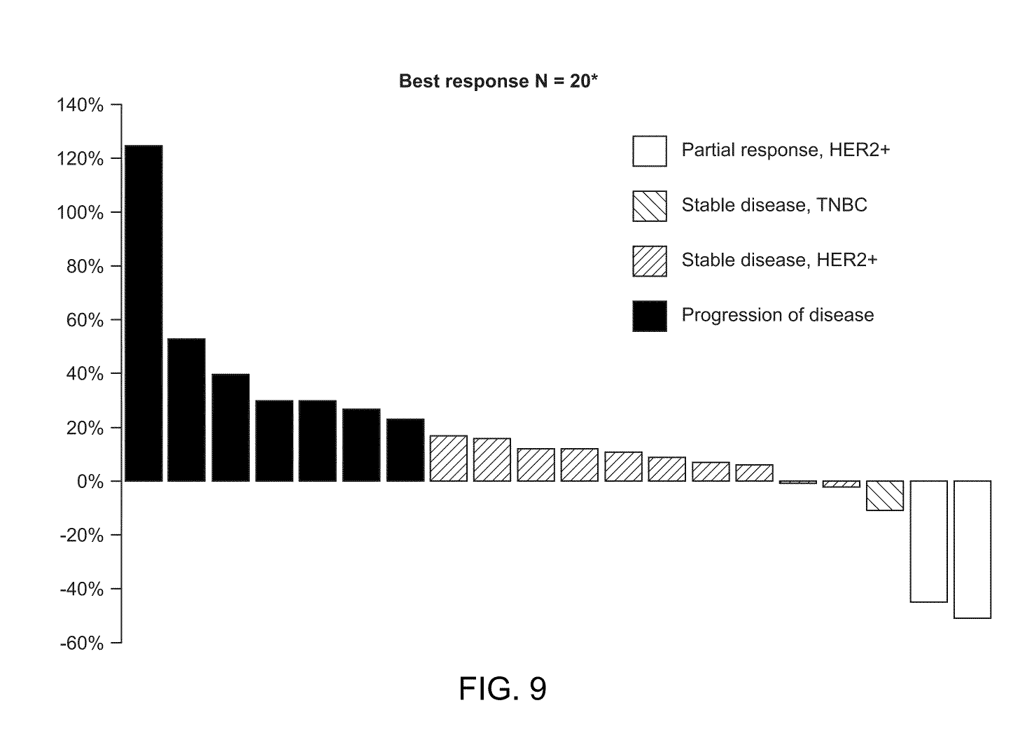

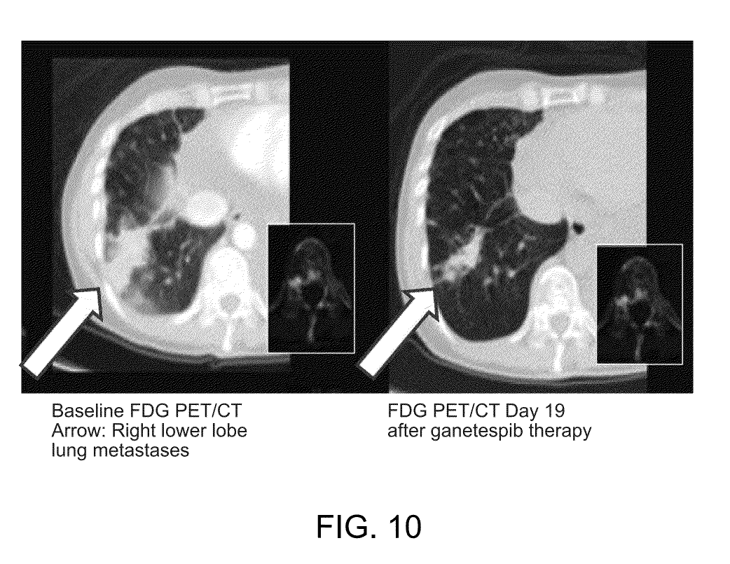

Female immunodeficient CD-1 (nude) mice (Charles River Laboratories, Wilmington, Mass.) were maintained in a pathogen-free environment, and all in vivo procedures were approved by the Synta Pharmaceuticals Corp. Institutional Animal Care and Use Committee. A549 NSCLC cells (7.5×10⁶) were subcutaneously implanted into the animals. Mice bearing established tumors (100-200 mm³) were randomized into treatment groups of 8 and i.v. dosed via the tail vein with either vehicle, ganetespib formulated in 10/18 DRD (10% DMSO, 18% Cremophor RH 40, 3.6% dextrose, 68.4% water) or p.o. dosed with BEZ235 formulated in PEG300/NMP (90% PEG300, 10% N-Methylpyrrolidone). Animals were treated with ganetespib at 50 mg/kg weekly or BEZ235 at 10 mg/kg 5 times a week, either alone or in combination. Tumor growth inhibition was determined as described previously. See Proia et al, PLoS One. 2011; 6(4):e18552. The results are shown in FIG. 8.

As shown in FIG. 8, average tumor volume was significantly reduced in mice treated with ganetespib and BEZ235 as compared to vehicle, particularly at later time points. The efficacy of ganetespib and BEZ235 were about the same, and average tumor volume substantially reduced in mice treated with ganetespib and BEZ235 as compared to vehicle control, particularly at later time points.

In summary, ganetespib displayed anticancer activity in all four breast cancer subtypes, as well as inflammatory breast cancer. Importantly, ganetespib was equally effective in killing cells grown as three dimensional spheres compared to cells grown in monolayer, as well as in vivo.

### Example 10

### Ganetespib Displays Activity Across GIST Subtypes

Many of the oncoproteins associated with gastric cancer and Hsp90 client proteins including HER2, MET, RAS and the FGFR family. Ganetespib was evaluated for its effect on the growth of AGS (wt-p53 and mut-KRAS) and MKN45 (wt-p53, wt-KRAS, MET amplified) gastric cancer cells. Cells were treated for 72 hr and viability determined by CTG (upper) or Syto60 (lower). Most gastric cell lines displayed low nanomolar IC50 with ganetespib, as shown in the Table below.

| | AGS | MKN45 |
|---|---|---|
| IC50 (nM) | wt-p53, mut-KRAS | wt-p53, wt-KRAS |
| Ganetespib | 1.5 | 0.5 |
| Docetaxel | 0.3 | 0.1 |
| 17-AAG | 50 | 1.0 |

| Cell Line | ganetespib IC50 (nM) |
|---|---|
| AGS | 5 |
| SNU-1 | 6 |
| GT3TKB | 6 |
| MKN-45 | 8 |
| GCIY | 9 |
| HGC-27 | 11 |
| ECC12 | 12 |
| SNU-5 | 31 |
| Hs746T | 384 |

Ganetespib was also evaluated for its effects on Hsp90 client proteins in AGS gastric cancer cells by western blot. Ganetespib abolished the expression of EGFR, IGF-IR, C-RAF and their down-stream effectors PI3K/AKT and MAPK, resulting in PARP cleavage and increased levels of p-Histone H2X (Ser139), a marker for DNA fragmentation during apoptosis. Similar to the observation in melanoma cells, exposure to ganetespib enhanced BRAF expression. Without being bound by mechanism, it is suggested that a decrease in phosphorylation of CDK1 by ganetespib may be due to the loss of WEE1 expression, an important regulatory kinase for CDK1. In MKN45 cells, exposure of ganetespib led to the complete degradation of MET and IGF-IR, followed by inactivation of AKT.

In summary, ganetespib displayed potent anticancer activity with low nanomolar IC50s in gastric cancer cell lines. Without being bound by mechanism, it is suggested that the activity is at least, in part, a result of widespread degradation of client proteins essential for cell growth, proliferation and survival including MET, IGF-1R, EGFR, WEE1 and CDK1.

### Example 11

### Ganetespib Displays Efficacy in Head and Neck Cancer Subtypes

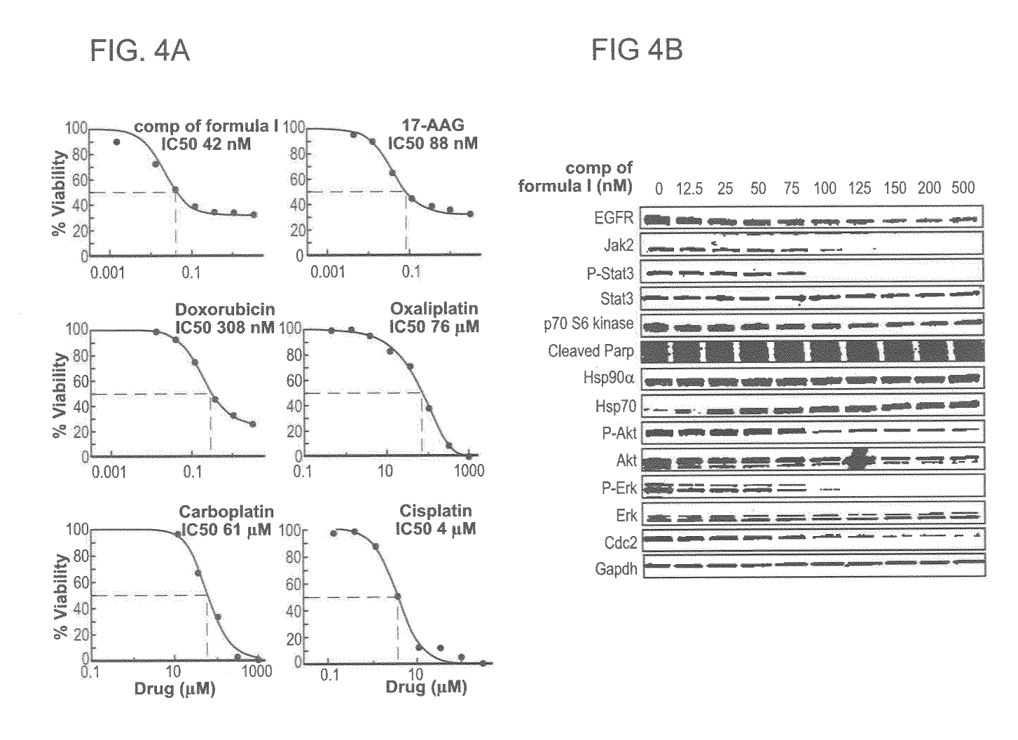

Head and neck (H/N) cancer refers to a group of biologically similar cancers originating from the upper autodigestive tract. First line therapies include EGFR inhibitors and platins. Modulation of EGFR and other client proteins by ganetespib was investigated in Detroit 562 H/N cancer cells. As shown in FIG. 4B, ganetespib led to the depletion of EGFR and JAK2, resulting in the inactivation of several key effectors including AKT, STAT3, p70S6, and ERK followed by cleaved PARP. Single agent viability analyses were then performed and it was found that the IC50 of ganetespib (42 nM) correlated with initiation of client protein degradation (FIG. 4A). A fraction of cells remained viable after a 72 hr exposure to ganetespib, in contrast to the platins which completely killed the cells.

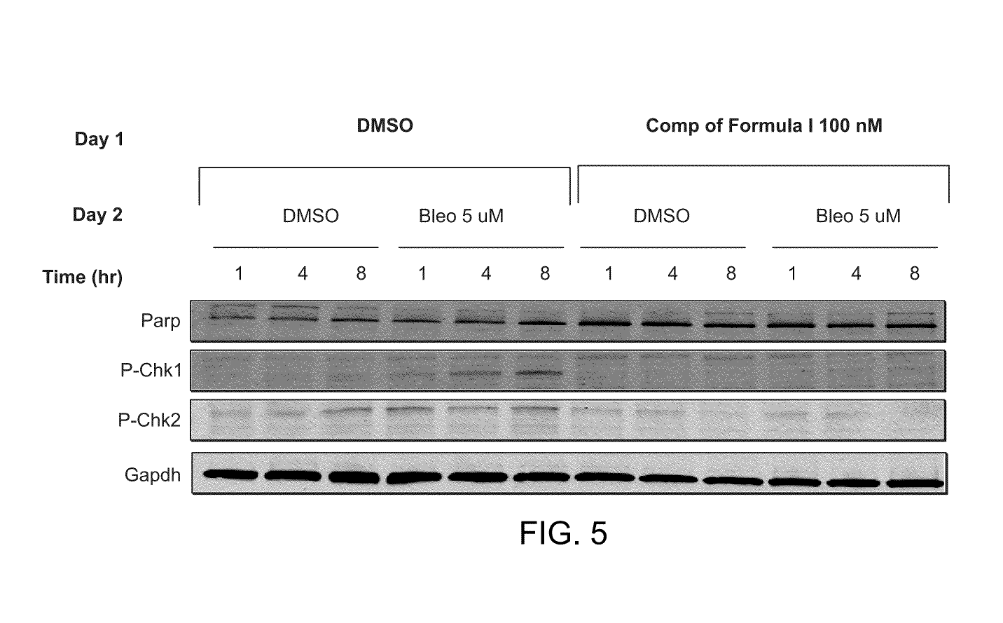

Western blot of protein expression is shown in FIG. 5. Cell extracts from Detroit 562 head and neck cancer cells were treated with 100 nM of ganetespib 24 hours prior to receiving the DNA damaging agent bleomycin (5 μM). Protein expression was measured at the indicated time points after bleomycin treatment. Bleomycin increased both Chk1 and Chk2 phosphorylation, which was blocked when cells were treated first with ganetespib.

### Example 12

### Ganetespib in Combination with Standard of Care Chemotherapies Displays Efficacy in NSCLC Cancer Subtypes with KRAS Mutations

Mutant KRAS is detected in 20-25% of non-small cell lung carcinomas (NSCLC) and represents one of the most common oncogenic drivers of this disease. NSCLC tumors with oncogenic KRAS respond poorly to currently available therapies necessitating the pursuit of new treatment strategies. Recent results from a Phase 2 trial with ganetespib revealed that >60% of patients with NSCLC having a KRAS mutation exhibited tumor shrinkage at 8 weeks, indicating that ganetespib is useful in the treatment of this disease.

To further understand the actions of ganetespib in NSCLC tumors having a KRAS mutation, studies were executed in a diverse panel of KRAS mutant NSCLC cell lines to investigate whether ganetespib is effective in suppressing critical cell signaling nodes responsible for KRAS-driven NSCLC cell survival and to assess whether ganetespib can synergize with both clinical agents targeted against these signaling nodes and standard of care chemotherapies.

For combinatorial analysis, cells were seeded in 96-well plates at a predetermined, optimum growth density for 24 h prior to the addition of drug or vehicle to the culture medium. Drug combinations were applied at a non-constant ratio over a range of concentrations for 72 or 96 hours. For each compound tested, a 7 point dose range was generated based on 1.5 fold serial dilutions using IC50 values set as the mid-point. Cell viability was assessed by either AlamarBlue® (Invitrogen, Carlsbad, Calif.) or CellTiter-Glo® assays and normalized to vehicle controls. For each combination study, the level of growth inhibition (fraction affected) is plotted relative to vehicle control. Data are presented as one relevant combination point and the corresponding single agent data for each cell line tested.

Ganetespib displayed potent anticancer activity across 15 KRAS mutant NSCLC cell lines assayed in vitro, with an average IC50 of 24 nM. Combining ganetespib with anti-mitotics, alkylating agents or topoisomerase inhibitors resulted in an increase in cell death of up to 44, 61 and 26%, respectively, versus monotherapy. At the molecular level, ganetespib induced the destabilization of several KRAS substrates, including BRAF and CRAF, leading to inactivation of their downstream effectors followed by programmed cell death. Ganetespib effectively suppressed the growth of human KRAS mutant NSCLC tumor xenografts in vivo; however, ganetespib did not induce tumor regression. In light of this, we sought to investigate whether inhibitors targeting KRAS driven signaling nodes would confer greater sensitivity to ganetespib. In vitro, combinations of low dose of ganetespib with either MEK or PI3K/mTOR inhibitors consistently resulted in greater activity than monotherapy, up to 77% and 42%, respectively. Furthermore, ganetespib suppressed activating feedback loops that occur in response to MEK and PI3K/mTOR inhibition, providing a rationale for the enhanced combinatorial activity. To validate these results, in vivo combinations were performed with ganetespib and a PI3K/mTOR inhibitor in KRAS mutant NSCLC xenografts. While both agents promoted tumor shrinkage on their own, considerable improvement in tumor growth inhibition was observed in the combination arm.

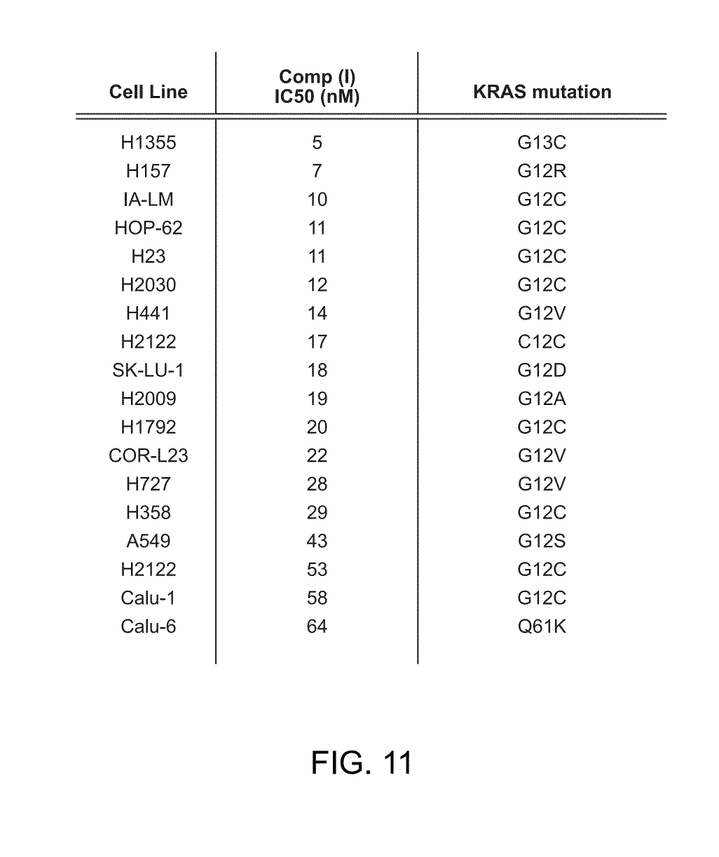

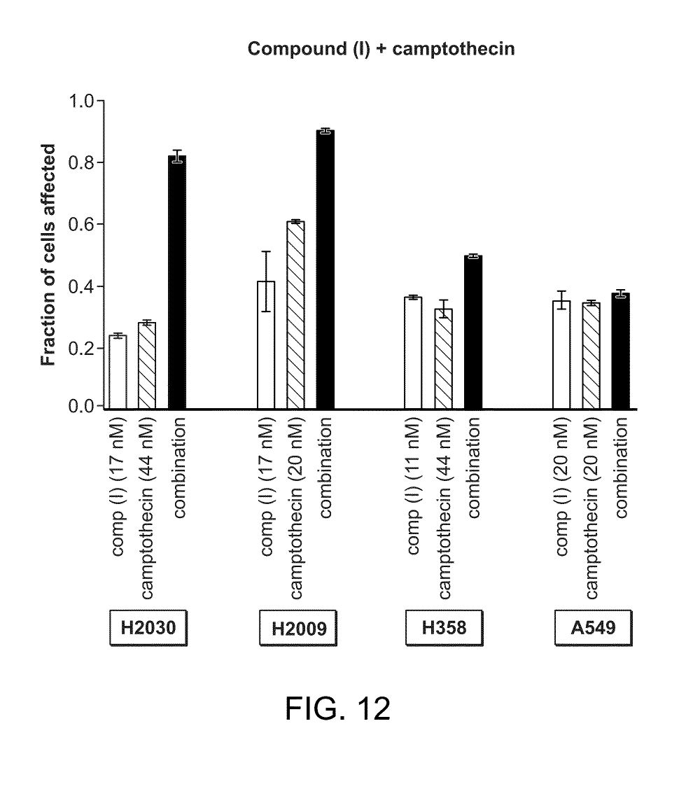

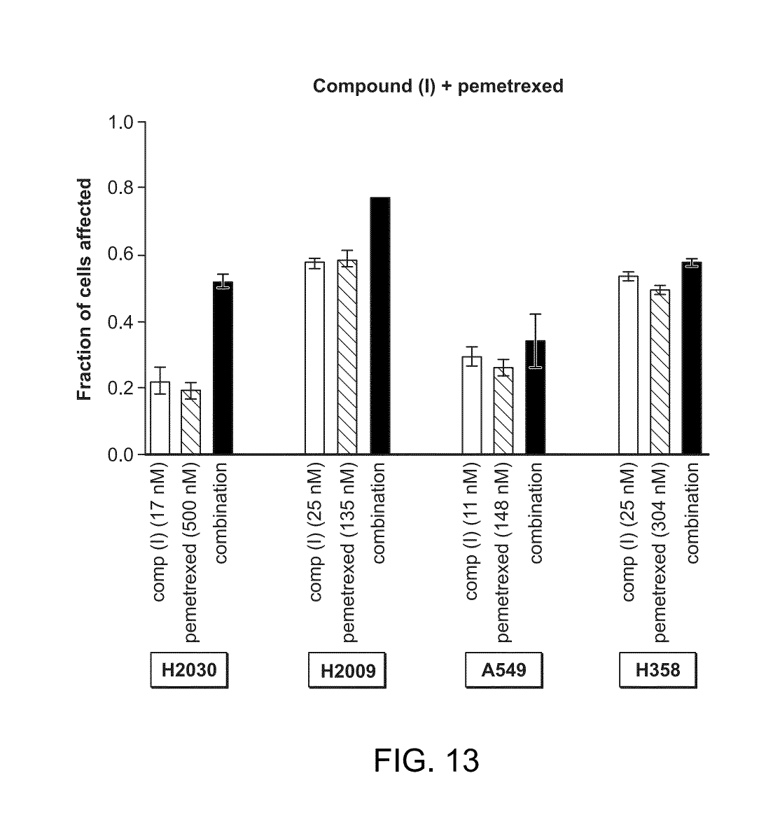

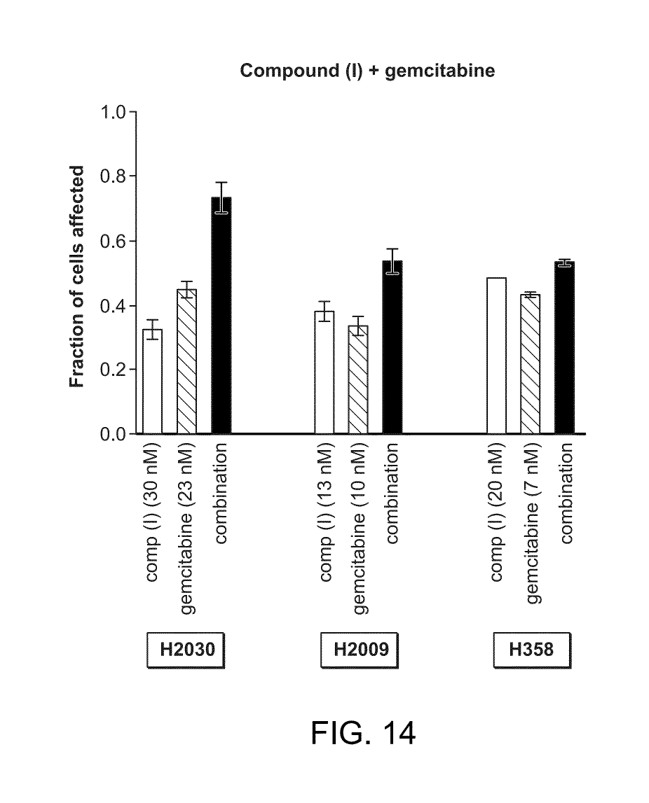

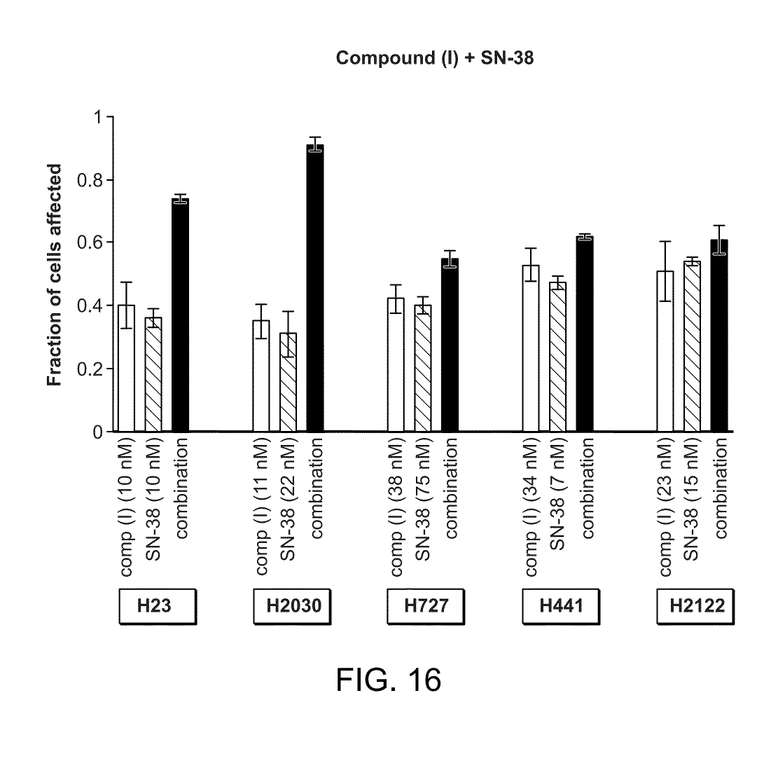

More particularly, ganetespib elicited promising activity against mutant KRAS NSCLC tumor cells (FIG. 11). In order to further identify feasible strategies to enhance the anti-tumor activity of ganetespib, combination studies were performed with standard of care chemotherapies in mutant KRAS NSCLC cell lines. Combining low nanomolar concentrations of ganetespib with the topoisomerase I inhibitor camptothecin resulted in a 1.5, 3.4, and 1.4 fold increase in cytotoxicity for H2009, H2030, and H358 cells, respectively (FIG. 12). Similar results were observed for SN-38, another topoisomerase I inhibitor (FIG. 16). It was also found that combining ganetespib with the antimetabolite pemetrexed enhanced cell death by 2.4 and 1.5 fold for H2030 and H2009 cells, respectively, while a marginal increase in cytotoxicity was observed for A549 and H358 cells (FIG. 13). Ganetespib in combination with the nucleoside analog, gemcitabine, increased cell death 2.3 and 1.4 fold for H2009 and A549 cells, respectively, and no benefit was observed for H358 cells (FIG. 14).

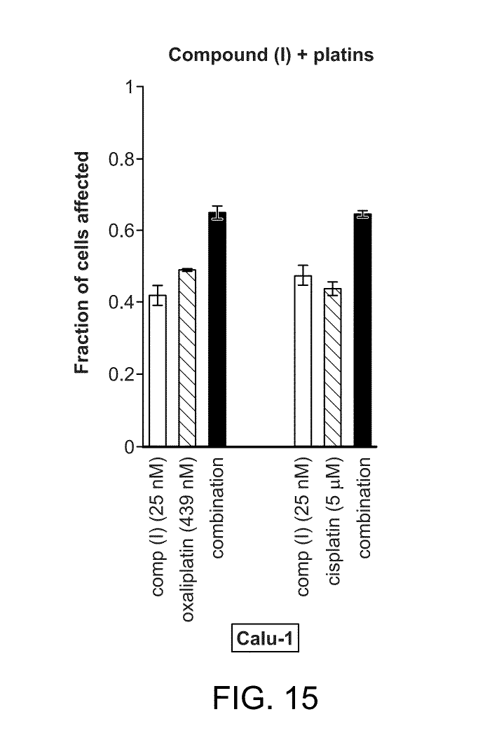

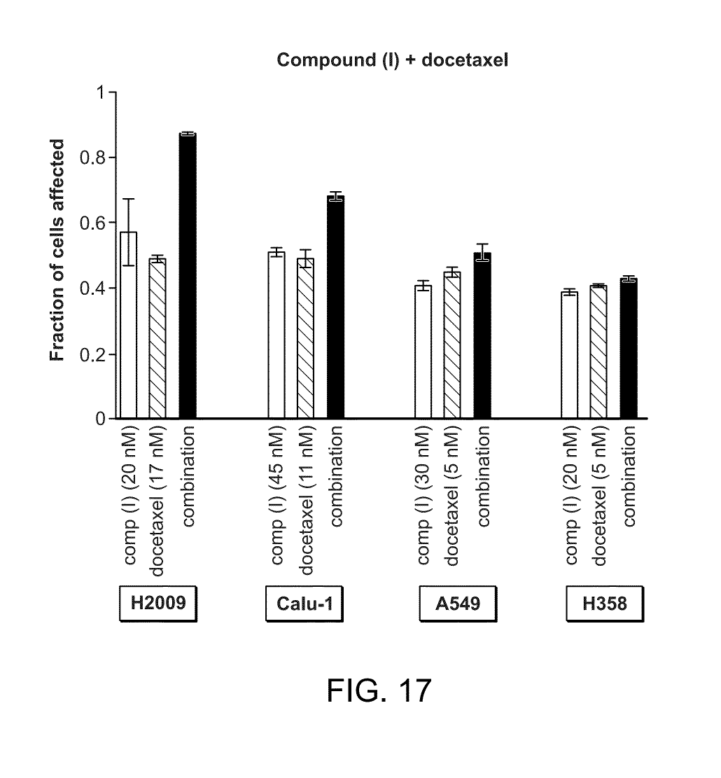

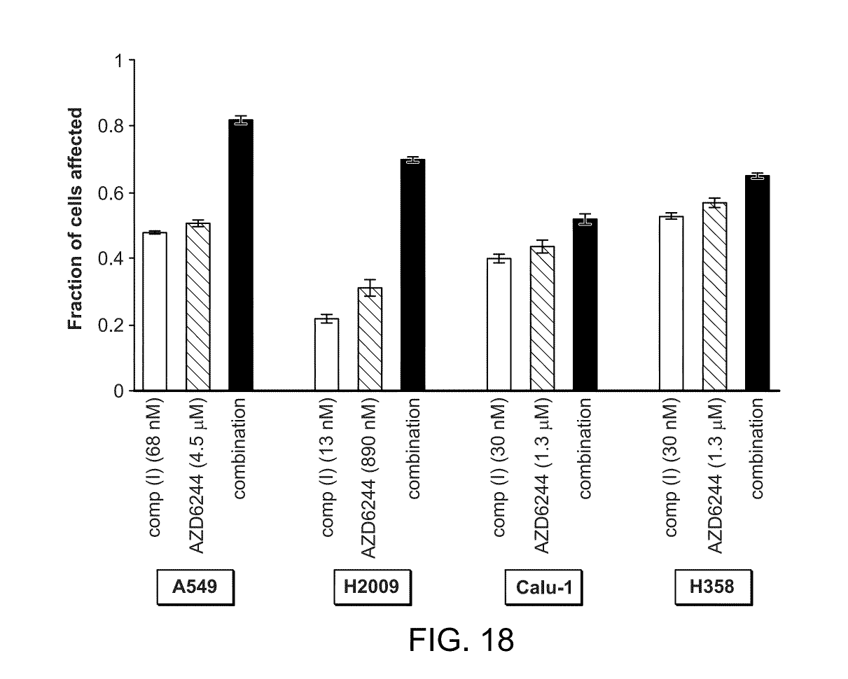

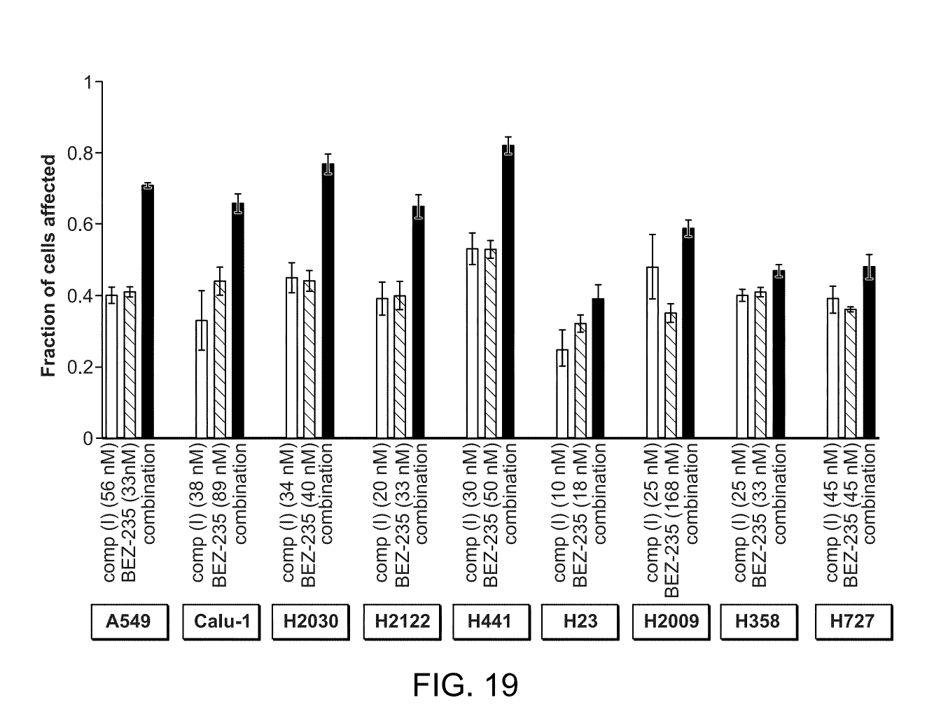

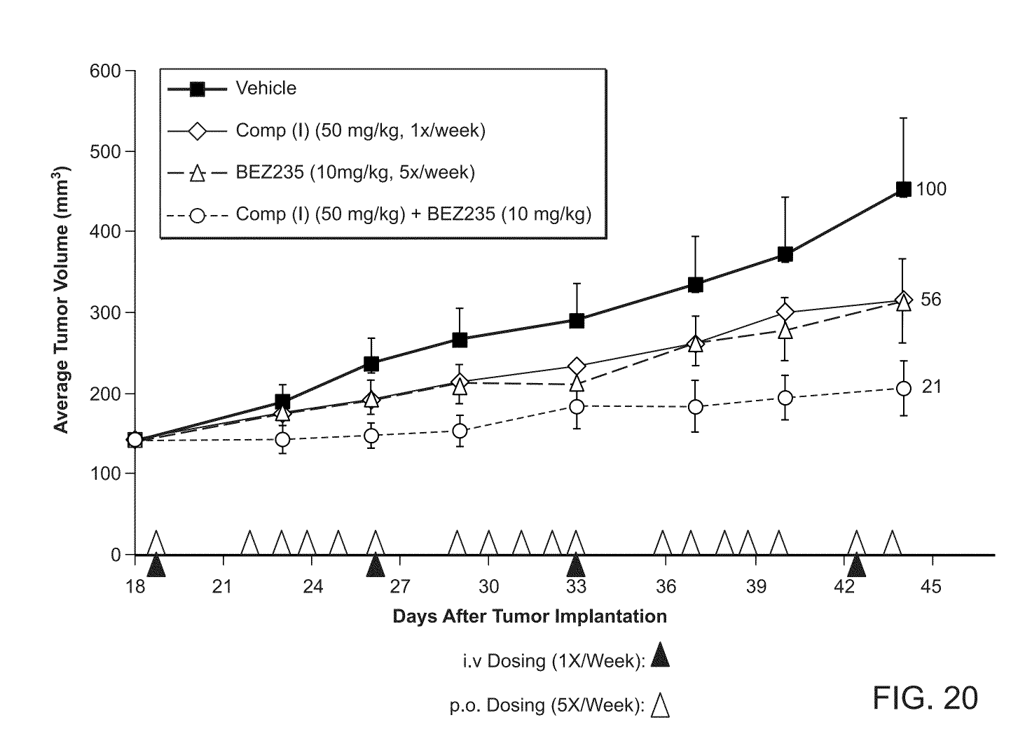

More combination data are presented in FIGS. 15-20. The results highlight the heterogeneity in response to various targeted agents and chemotherapies as well as the variability in benefit achieved when these agents are combined with ganetespib. Taken together, these results suggest that chemotherapies currently used for the treatment of NSCLC may enhance the antitumor activity of ganetespib.

Without being bound by mechanism, it is suggested that ganetespib promotes destabilization of multiple oncogenic signaling proteins and is potently cytotoxic in KRAS mutant NSCLC cells and simultaneously disrupts multiple nodes of KRAS driven signaling resulting in enhanced apoptosis compared to MEK or PI3K/mTOR inhibitors. Combining ganetespib with MEK or mTOR inhibitors blocks feedback induced accumulation of activated MEK and ERK contributing to enhanced cytotoxicity in vitro and in vivo. Common standard of care chemotherapeutics utilized in the treatment of NSCLC enhance the activity of ganetespib.

In summary, ganetespib, a potent inhibitor of Hsp90, has shown encouraging evidence of clinical activity, including tumor shrinkage in patients with KRAS mutant NSCLC. In vitro, ganetespib exhibited potent anticancer activity in NSCLC cells with a diverse spectrum of KRAS mutations due in part to degradation and inactivation of critical KRAS signaling effectors. Combination with targeted therapies that overlap with these signaling nodes led to enhanced anticancer activity in vitro and in mouse models of KRAS mutant NSCLC. Taken together, these results demonstrate clinical utility of ganetespib in patients with KRAS mutant NSCLC.

Standard of care chemotherapeutics utilized in KRAS mutant NSCLC show activity with ganetespib in vitro. Camptothecin, pemetrexed and gemcitabine showed up to 4 fold increases in cell death when combined with ganetespib. None of the agents antagonized the anticancer activity of ganetespib.

### Example 13

### Phase 1 Trial of the Combination of Ganetespib and Docetaxel in the Treatment of Solid Tumors

A Phase 1 study of ganetespib in combination with docetaxel in solid tumors has been studied in a broad range of clinical trials.

A trial to evaluate three dose-level combinations of docetaxel and ganetespib, administered on a three-week cycle, with the primary objective of determining an optimal dose for future clinical trials was performed. Docetaxel was administered as a one hour IV infusion on day 1 and ganetespib was administered as a one hour IV infusion on days 1 and 15. The dose level combinations evaluated were 150 mg/m² and 60 mg/m²; 150 mg/m² and 75 mg/m²; and 200 mg/m² and 75 mg/m² for ganetespib and docetaxel respectively. The standard of care dose level for docetaxel was 75 mg/m². A total of 19 patients received at least one dose of study treatment at the cut-off time. The median number of cycles of treatment was 4, with a range of 1 to 11 cycles of treatment. No prophylactic treatment for neutropenia was used. The combination of ganetespib at 150 mg/m² and docetaxel at 75 mg/m² was selected as the recommended dose.

It was observed that a patient responded with over 50% shrinkage of target tumor lesions on the trial diagnosed with cancer of the parotid gland, the largest of the salivary glands. The patient did not respond to prior treatment regimens including carboplatin, cetuximab, and methotrexate.

The most common adverse event was neutropenia (67%), including four patients (22%) who reported febrile neutropenia. Neutropenia, a known effect of docetaxel treatment, was commonly observed at approximately 8 days following dosing and typically resolved spontaneously within 7 days. Serious adverse events were reported in a total of nine patients (50%) including two reports of pneumonia and one report each of chest pain, chills, dyspnea, fatigue, mucosal inflammation, neutropenia, pneumothorax, pulmonary embolism, rib fracture, transient ischaemic attack, and vomiting.

Pharmacokinetic data indicate a pharmacokinetic similarity between ganetespib administered alone and ganetespib administered prior to docetaxel. There was no effect of ganetespib on docetaxel pharmacokinetics.

These results support the use of ganetespib at a dose of 150 mg/m² in combination with docetaxel at a dose of 75 mg/m² for treating NSCLC and other solid cancers.

### Example 14

### Method of Evaluating Activity Levels of LDH Isoforms in Subject Samples

Human tumor cell lines HCT116 (ATCC #CRL-247; Schroy P C, et al. Cancer 76: 201-209, 1995) and 786-0 (ATCC #CRL-1932; Williams R D, et al. In Vitro 12: 623-627, 1976), were obtained from the American Type Culture Collection (Manassas, Va., USA) were cultured using routine methods until a sufficient number of cells were obtained for implantation. Studies were conducted on animals between 7 and 12 weeks of age at implantation. To implant HCT116 tumor cells into nude mice, the cells were trypsinized, washed in PBS and resuspended at a concentration of 75×10⁶ cells/ml in McCoy's modified medium with 50% of BD Matrigel® Basement Membrane Matrix (BD Biosciences®, Bedford, Mass., USA). To implant 786-0 tumor cells into nude mice, the cells were trypsinized as above, washed in PBS and resuspended at a concentration of 75×10⁶ cells/ml in RPMI 1640 medium with 50% of BD Matrigel® Basement Membrane Matrix. Using a 27 gauge needle and 1 cc syringe, 0.1 ml of the cell suspension was injected into the corpus adiposum of nude mice. The corpus adiposum is a fat body located in the ventral abdominal vicera in the right quadrant of the abdomen at the juncture of the os coxae (pelvic bone) and the os femoris (femur). The location permits palpation and measurement of the tumors using external calipers. Tumor volumes (V) were calculated by caliper measurement of the width (W), length (L) and thickness (T) of tumors using the following formula: V=0.5236×(L×W×T). Animals were randomized into treatment groups so that the average tumor volumes of each group were similar at the start of dosing.

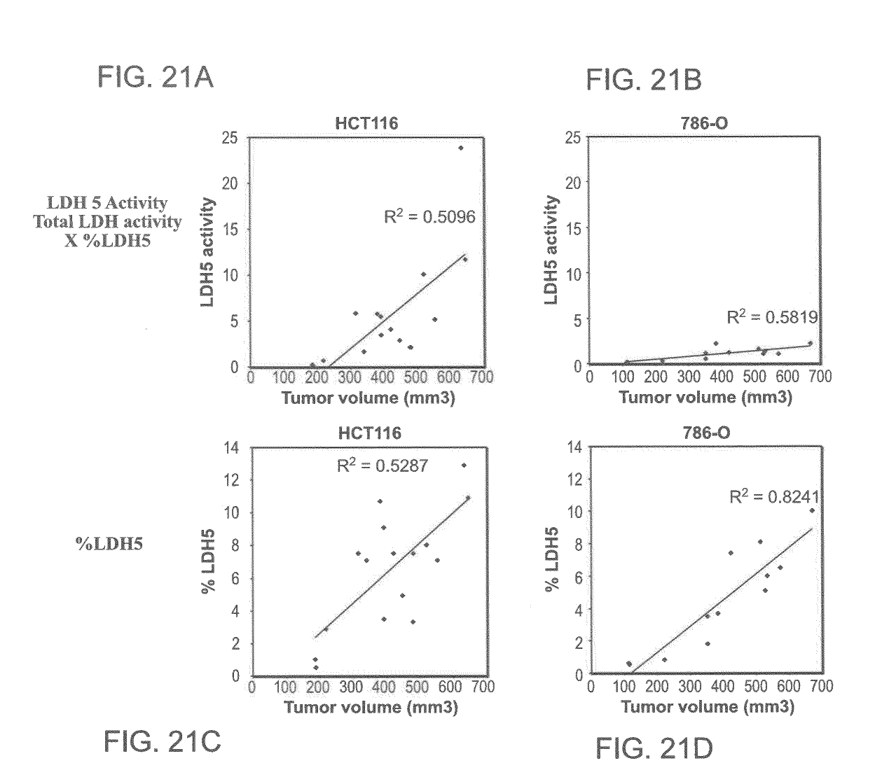

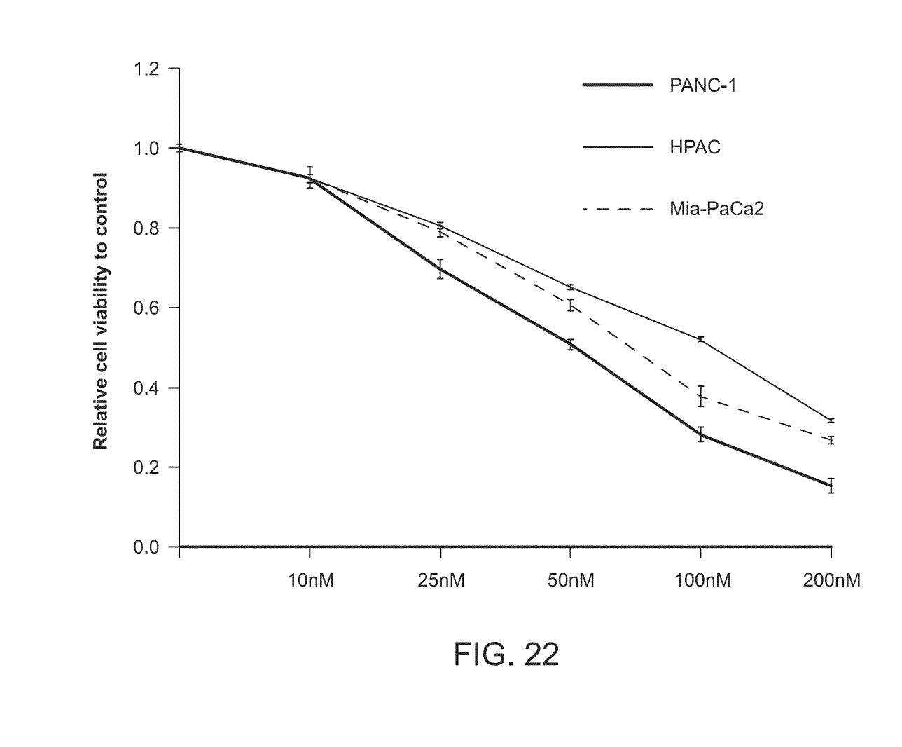

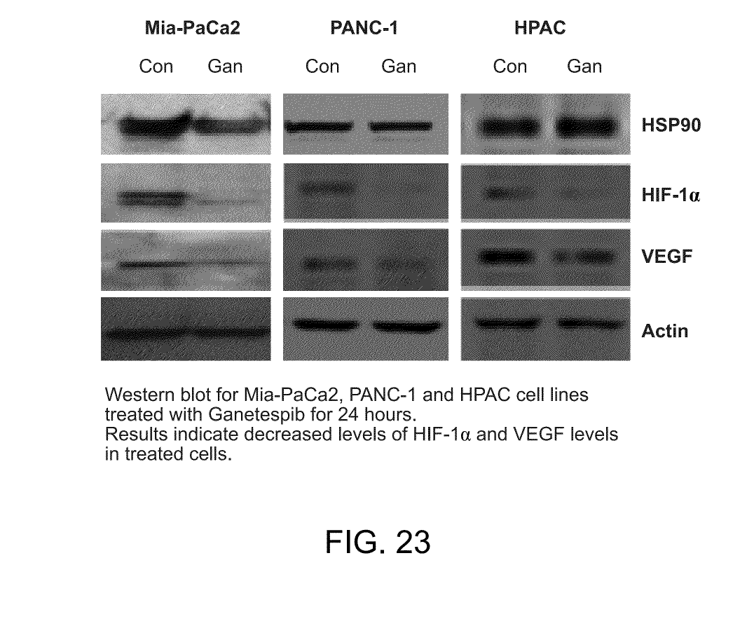

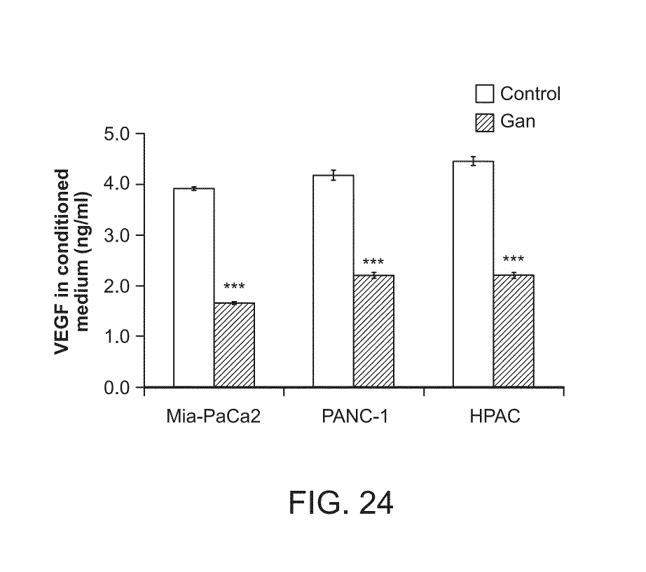

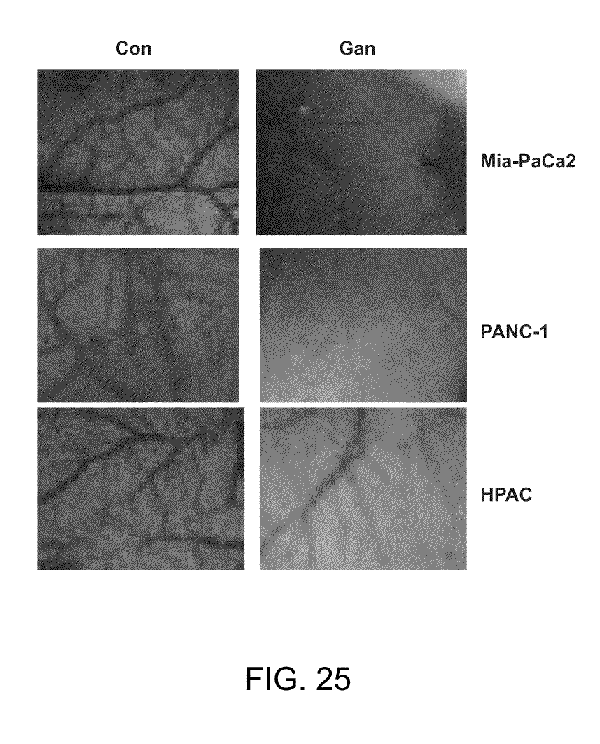

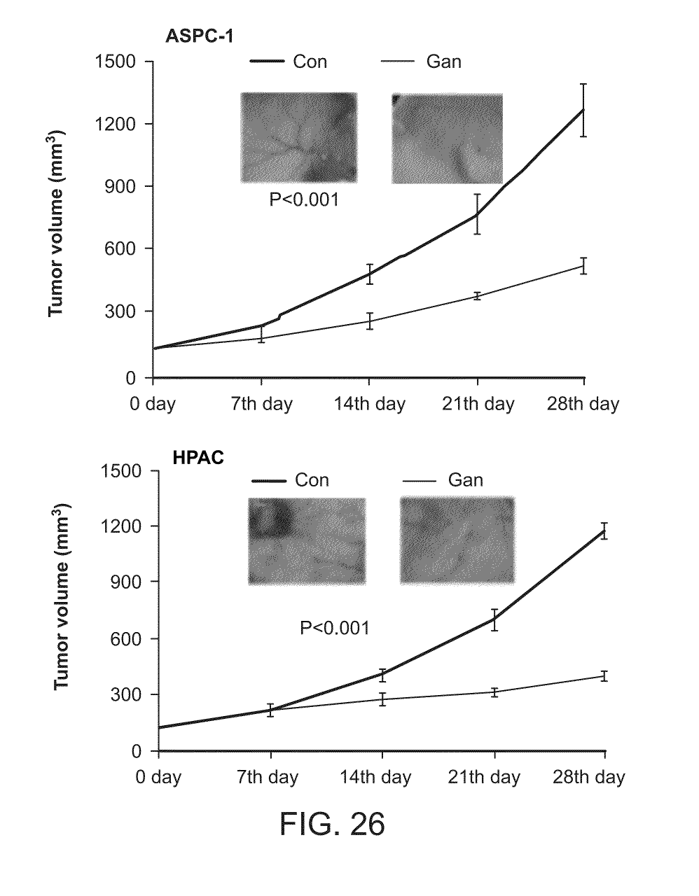

Blood was collected from the tumor bearing mice at appropriate time points, serum was prepared, and the serum frozen for later analysis. On the same days as blood collection, tumor volumes (V) were calculated by caliper measurement of the width (W), length (L) and thickness (T) of tumors using the following formula: V=0.5236×(L×W×T). After collection of the serum samples was completed, serum samples were resolved by gel electrophoresis. Following electrophoresis, the bands for the five isoenzymes were visualized by an enzymatic reaction using an in-gel assay. Lactate, nicotinamide adenine dinucleotide (NAD+), nitroblue tetrazolium (NBT), and phenazine methosulphate (PMS) were added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of LDH5 was determined by densitometry (Beckman Appraise densitometer, Beckman Coulter Inc. or Sebia (GELSCAN, Sebia Inc). The percent of LDH5 protein and LDH5 activity relative to the total LDH present (i.e., the amount of LDH5, LDH5, LDH3, LDH2, and LDH1 combined) was calculated and graphed against tumor volume. The results are shown in FIGS. 21A-D.

FIGS. 21A and 21B show the amount of LDH5 activity as a percent of total LDH activity as determined by the in-gel assay. As shown, the HCT116 tumors had a substantially greater percent to LDH5 activity relative to total LDH activity as compared to the 786O tumors. FIGS. 21C and 21D demonstrate that despite the difference in the relative activity of LDH5 that is observed, the amount of LDH5 protein present relative to total LDH is about the same for both tumor types.

### Example 15

### Selection of Subjects for Treatment with an Hsp90 Inhibitor Based on a Level of Hypoxia

Multiple clinical trials for the treatment of cancer have been performed using Hsp90 inhibitor compounds, e.g., ganetespib such as those described herein. Such studies can be performed to analyze the effect of the level of hypoxia on treatment outcomes with Hsp90 inhibitors such as ganetespib.

In such studies, a subject is diagnosed with a cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a high level of a hypoxic marker in serum and/or in the tumor. The subject is selected for treatment with the Hsp90 inhibitor, e.g., ganetespib, known to be effective in treating cancer in a subject having a high level of hypoxic marker. The subject is treated with tan Hsp90 inhibitor, and monitored for therapeutic response as well as the presence of side effects. Therapy is continued as long as it is sufficiently tolerated and a benefit to the subject is observed as determined by the subject, the treating physician, the caregiver, and/or other qualified individual.

### Example 16

### Selection of Subjects not to be Treated with an Hsp90 Inhibitor Based on a Level of Hypoxia

A subject is diagnosed with cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a low level of a hypoxic marker in serum and/or in the tumor. A treatment regimen not including an Hsp90 inhibitor known to be effective in treating cancer in a subject having a high level of hypoxic marker is selected for the subject.

### Example 17

### Characterization of Treatment Outcomes with an Hsp90 Inhibitor Based on Chart Review

A chart review analysis is performed to determine the efficacy of an Hsp90 inhibitor, e.g., ganetespib, for the treatment of a cancer based on the level of hypoxia of the tumor based on markers assessed during the treatment of the subjects. Inclusion criteria are information being available regarding the cancer type, the specific treatment regimen with the Hsp90 inhibitor, and the outcome over a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up with subjects (e.g., until tumor progression, until administration of new therapeutic intervention, to an arbitrary end point). In addition to information related to survival, information related to quality of life, side effects, and other relevant information is considered when available. Exclusion criteria can include the presence of other diseases or conditions that could result in alteration of levels of hypoxia-modulated peptides, e.g., ischemic heart or vascular disease, poor circulation, diabetes, macular degeneration, recent stroke, recent surgery, or other ischemic events or conditions. Other exclusion criteria can be selected based on the available samples and patient population, e.g., prior treatment with specific agents.

The subjects can be sorted into groups based on various criteria. Subjects who were treated with an Hsp90 inhibitor, e.g., ganetespib, for whom no levels of hypoxic markers were determined can be used as an unstratified control group to understand the efficacy of the Hsp90 inhibitor on a treatment population not selected based on the level of hypoxia in the subject/tumor. Alternatively, the population analyzed in the study for which hypoxia levels (e.g., LDH marker levels) can be compared to historical control samples in which an unstratified population was analyzed for response to the agent.

Subjects for whom hypoxic levels are available in chart records are divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with the selected agent, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), and ornithine decarboxylase (ODC). Antibodies against prodrugs that localize in hypoxic regions (e.g., EF5, pimonidazole, etc.) can also be markers hypoxia. Functional imaging measuring blood flow in the tumor can be used as a marker of hypoxia in the tissue. Direct measurement of hypoxia can be a marker and can be performed by inserting a sensor into the tumor. Tumor size can also be a marker correlated with hypoxia. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be performed by inserting a sensor into the tumor. Tumor size can also be a marker for hypoxia. Again, it is preferred that the same method of determining the level of the marker of hypoxia is used for all samples, particularly when qualitative assessment methods are used.

Outcomes of subjects based on the level of hypoxia are analyzed to determine if the outcome between the two groups is different. Outcomes can further be compared to a non-stratified group treated with the Hsp90 inhibitor. Methods for statistical analysis and determination of statistical significance are within the ability of those of skill in the art. For the Hsp90 inhibitor, the analysis demonstrates that subjects with a high level of hypoxia have a better response, e.g., one or more of longer time to failure, longer survival time, better quality of life, decreased tumor size, better tolerance of the selected agent, etc., as compared to subjects with a low level of hypoxia, and that Hsp90 inhibitors should be preferentially used in subjects having high levels of markers of hypoxia.

### Example 18

### Characterization of Treatment Outcomes with an Hsp90 Inhibitor Based on Historical Samples

An analysis using samples collected from subjects during treatment is performed to determine the efficacy of an Hsp90 inhibitor, e.g., ganetespib, for the treatment of cancer based on the level of hypoxia of the tumor based on markers assessed prior to and/or during the treatment of the subjects. Inclusion criteria are information being available regarding the cancer type, the specific treatment regimen with the selected agent, and the outcome for a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up (e.g., until tumor progression, until administration of new therapeutic intervention, to an arbitrary end point) with subjects. In addition to information related to survival, information related to quality of life, side effects, and other relevant information is considered when available. Exclusion criteria include the presence of other diseases or conditions that could result in alteration of levels of hypoxia-modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with the Hsp90 inhibitors.

The samples are analyzed for the level of hypoxia. Preferably, all of the samples are the same type or types, e.g., blood, plasma, lymph, urine, tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia are measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, neurolipin 1 (NRP-1), pyruvate dehydrokinase (PDH-K), and ornithine decarboxylase (ODC), tumor size. Antibodies against prodrugs that localize in hypoxic regions (e.g., EF5, pimonidazole, etc.) can also be markers hypoxia. Functional imaging measuring blood flow in the tumor can be used as a marker of hypoxia in the tissue. Direct measurement of hypoxia can be a marker and can be performed by inserting a sensor into the tumor. Tumor size can also be a marker correlated with hypoxia. Further, it is preferred that the same type of subject sample, e.g., blood, serum, lymph, urine, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be performed by inserting a sensor into the tumor. Tumor size can also be a marker for hypoxia. Again, it is preferred that the same method of determining the level of the marker of hypoxia was determined using the same method in all samples, particularly when qualitative assessment methods are used.

Subjects are divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with an Hsp90 inhibitor, cancer type, previous failed treatments, etc. for analysis.

Outcomes of subjects based on the level of hypoxia are analyzed to determine if the outcome between the two groups is different. Outcomes can further be compared to a non-stratified group treated with an Hsp90 inhibitor e.g., a historical group provided by another study. Methods for statistical analysis and determination of statistical significance are within the ability of those of skill in the art. For the Hsp90 inhibitor, the analysis demonstrates that subjects with a high level of hypoxia have a better response, e.g., one or more of longer time to failure, longer survival time, better quality of life, decreased tumor size, better tolerance of the selected agent, delayed time to progression, etc., as compared to subjects with a low level of hypoxia, and that such Hsp90 inhibitors should be preferentially used in subjects having high levels of markers of hypoxia.

### Example 19

### Trial to Demonstrate Improved Efficacy of an Hsp90 Inhibitor in Subjects with a Modulated Level of Hypoxia

Subjects diagnosed with solid tumors are recruited for a study to determine the efficacy of an Hsp90 inhibitor, e.g., ganetespib, in the treatment of solid tumors, preferably tumors from the same tissue origin, e.g., breast, prostate, lung, liver, brain, colorectal, etc. Inclusion criteria include the presence of a solid tumor and at least 30 days from surgery and any incisions are fully closed. Exclusion criteria include the presence of an ischemia related disease or disorder including, e.g., ischemic heart or vascular disease, poor circulation, diabetes, macular degeneration, recent stroke, or other ischemic events or conditions; or surgery planned during the duration of the trial. Blood and tumor samples are collected for analysis of levels of hypoxia by determining the level of one or more markers of hypoxia, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), ornithine decarboxylase (ODC), tumor size. Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be performed by inserting a sensor into the tumor. Tumor size can also be a marker for hypoxia. Depending on the tumor site, other subject samples can be collected, e.g., fecal matter in subjects with colorectal cancer, urine for subjects with kidney or bladder cancer, cerebrospinal fluid in subjects with brain cancer, etc. by assaying the same markers. Additional samples for analysis can be collected during the course of the study. Complete medical histories are also obtained when not otherwise available.

All subjects are treated with the Hsp90 inhibitor, either alone or in combination with one or more additional chemotherapeutic agents. The number regimens used will depend on the size of the study, the number of subjects available, the time frame of the study, etc. The number of regimens is selected to allow the study to be sufficiently powered to provide meaningful results. Subjects are monitored for response to the agent throughout the trial, at the end of the trial, and at regular intervals after the conclusion of the trial using routine methods including, but not limited to, e.g., imaging, hematology, and physical examination. Treatment may be discontinued for non-responsive subjects or for with intolerable side effects. Preferably, the subjects continue to be monitored for outcomes beyond the formal end of the trial. Subjects with a positive response to the treatment regimen can be continued on the regimen beyond the predetermined treatment window of the trial at the discretion of the attending physician.

An analysis of the samples collected from subjects prior to and optionally during treatment is performed to determine the efficacy of the Hsp90 inhibitor for the treatment of cancer based on the level of hypoxia of the tumor based on markers assessed prior to and optionally during the treatment of the subjects. The analysis can be performed at the conclusion of the trial, or the analysis can be performed prior to the conclusion of the trial with the results being blinded or not disclosed to the treating physicians. Preferably, the analysis for hypoxia level is determined during the course of the trial to insure that a sufficient number of subjects with high and low hypoxia levels were enrolled in the study to allow for sufficient power of the study to provide a conclusive outcome.

Outcomes of subjects based on the level of hypoxia are analyzed to determine if the outcome between the two groups is different. Outcomes can further be compared to a non-stratified group treated with the agent, e.g., a historical group provided by another study. Samples can be analyzed to confirm the correlation of the level of hypoxia in the tumor to the level of hypoxia in the peripherally collected sample (e.g., blood, urine, cerebrospinal fluid). Methods for statistical analysis and determination of statistical significance are within the ability of those of skill in the art. The analysis demonstrates that subjects with a high level of hypoxia have a better response, e.g., one or more of longer time to failure, longer survival time, better quality of life, decreased tumor size, better tolerance of the selected agent, etc., as compared to subjects with a low level of hypoxia, and that such agents should be preferentially used in subjects having high levels of markers of hypoxia.

### Example 20

### Characterization of Treatment Outcomes to Demonstrate Improved Efficacy of Hsp90 Inhibitors in Subjects with Solid Tumors with a High Level of LDH

Clinical trials have been performed to demonstrate the efficacy of Hsp90 inhibitors, e.g., ganetespib, in the treatment of cancer. A chart review is performed to determine if levels of one or more hypoxic markers, particularly LDH, is analyzed for the subjects prior to, and optionally during treatment with ganetespib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with ganetespib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including Hsp90 inhibitors based on the ULN level. The outcome of the analysis is further used to allow for the selection of subjects likely to benefit from treatment with Hsp90 inhibitors based on the ULN level. Subjects with a high level of LDH are selected for treatment with Hsp90 inhibitors as they are likely to benefit from such treatment. Subjects with a low level of LDH are selected against for treatment with Hsp90 inhibitors as they are not likely to benefit from such treatment.

### Example 21

### Characterization of Treatment Outcomes to Demonstrate Improved Efficacy of Ganetespib in Subjects with Cancers with a High Level of LDH

Multiple Phase 1 and 2 clinical trials have been and are being performed to demonstrate the efficacy of ganetespib in non-small cell lung cancer, gastrointestinal stromal tumors, colorectal cancer, gastric cancer, small cell lung cancer, and melanoma as discussed in the previous example.

A chart review is performed to determine if levels of one or more hypoxic markers, particularly LDH, is analyzed for the subjects prior to, and optionally during treatment with ganetespib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with ganetespib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including ganetespib based on the ULN level. The outcome of the analysis is further used to allow for the selection of subjects likely to benefit from treatment with ganetespib based on the ULN level. Subjects with a high level of LDH are selected for treatment with ganetespib as they are likely to benefit from such treatment. Subjects with a low level of LDH are selected against for treatment with ganetespib as they are not likely to benefit from such treatment.

### Example 22

### Trial to Demonstrate Improved Efficacy of Hsp90 Inhibitors in Subjects with Various Cancer Types with a High Level of LDH

Subjects are identified as having one of advanced solid tumor malignancies including metastatic or unresectable malignancy with evidence of progression, non-small cell lung cancer, gastrointestinal stromal tumors, colorectal cancer, gastric cancer, small cell lung cancer, melanoma, refractory malignancy. A subject is selected as being candidate for treatment with an Hsp90 inhibitor, e.g. ganetespib. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of an Hsp90 inhibitor, either alone or in combination with other agents, e.g., using the regimens presented in the prior examples. At predetermined regular or irregular intervals, subjects are assessed for specific outcomes including, but not limited to, overall survival, progression free survival, time to progression, and adverse events. Treatment is continued for as long as the subject responds positively to treatment with the Hsp90 inhibitor and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including an Hsp90 inhibitor based on the ULN level. The outcome of the analysis is further used to allow for the selection of subjects likely to benefit from treatment with an Hsp90 inhibitor based on the ULN level. Subjects with a high level of LDH are selected for treatment with an Hsp90 inhibitor as they are likely to benefit from such treatment. Subjects with a low level of LDH are selected against for treatment with ganetespib as they are not likely to benefit from such treatment.

### Example 23

### Selection of Subjects with Lung Cancer and a High Level of LDH for Treatment with Ganetespib

Subject is identified as having lung cancer, either small cell or non-small cell lung cancer, or other cancer type known to be or suspected to be susceptible to treatment with ganetespib, and being candidate for treatment with ganetespib. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than ganetespib is selected. If the subject has a high LDH level, treatment with ganetespib, optionally with other agents, is selected as the treatment regimen.

### Example 24

### Antiangiogenic Activity of Ganetespib in Pancreatic Cancer Models

Pancreatic cancer is the fourth most common cause of cancer related mortality in US. In the year 2012 alone, approximately 43,900 new cases of pancreatic cancer are estimated in the US.

It's postulated that functional inhibition of Hsp90 by ganetespib can inhibit angiogenesis and growth in vitro and in vivo models of pancreatic cancer. PANC-1 and HPAC cell lines were treated with vehicle or G (50 nM) for 24 h and lysates were analyzed by Western blot. Egg CAM and matrigel plug assays were performed to quantify the effects of ganetespib on angiogenesis. Efficacy of ganetespib (100 mg/kg) was assessed in mice bearing HPAC and ASPC-1 xenograft. Western blot analyses demonstrated a significant reduction in intracellular HIF-1α a and VEGF protein levels in PANC-1 and HPAC cells treated with G. Results from ELISA assays showed that ganetespib reduced VEGF secretion in the culture medium from both pancreatic lines. Treatment of genetespib reduced angiogenesis compared to vehicle in all three models. Animals with human pancreatic tumor xenografts treated with ganetespib had significant tumor growth delay and inhibition of angiogenesis. The preclinical data demonstrates that ganetespib can inhibit pancreatic cancer growth and angiogenesis, suggesting that targeting Hsp90 is a rational new approach to pancreatic cancer therapy to be explored in clinical trials.

Materials and Methods

Cell Lines:

Mia-PaCa2, PANC-1, HPAC and ASPC-1 cell lines (ATCC, Manassas, Va.) were cultured according to the ATCC manual. Medium was supplemented with 10% fetal bovine serum (Invitrogen Corporation, Carlsbad, Calif.), 50 units/ml penicillin, and 50 μg/ml streptomycin (Life Technologies, Inc., Frederick, Md.). Cells were incubated at 37° C. in a humidified 5% CO₂ atmosphere.

Chemicals and Antibodies:

Primary antibodies specific to Hsp90, HIF-1α, VEGF, Actin and HRP conjugated secondary antibodies (Santa Cruz Biotechnology, California and Cell Signaling Technology, USA) were used for Western blot. Ganetespib was provided by Synta Pharmaceuticals, Lexington, Mass. and Matrigel was purchased from BD Biosciences.

VEGF Levels:

VEGF concentration in the conditioned medium (from control and treated cells) was determined using a commercial Human VEGF Quantikine ELISA kit (R&D systems, Minneapolis, Minn.) as per manufacturer's instructions.

Western Blotting:

Cells were harvested at the end of treatment and lysed in the RIPA protein extraction buffer (Sigma-Aldrich, Saint Louis, Mo., USA) containing the protease inhibitor cocktail. Equal amounts of protein fractions of lysates were resolved over SDS-PAGE and transferred on to PVDF membrane. Membranes were incubated with primary antibodies followed by HRP-conjugated secondary antibodies. Bound antibodies were visualized using enhanced chemiluminescence. To confirm equal loading, membranes were verified and re-probed with an antibody specific for the housekeeping gene, anti-β-actin.

Egg Cam Assay:

Mia-PaCa2, PANC-1 and HPAC cells were treated with Ganetespib (50 nM) or control for 24 hours, the conditioned medium was harvested and 100 μl of conditioned or control medium was injected into fertilized chicken eggs (Avian Vaccine Service center, North Franklin, Conn.). Eggs were incubated at 37° C. for 15 days and dissected. Chorioallantoic membrane was photographed.

In vivo tumor growth delay and angiogenic assay: Five-week-old SCID mice were divided into 4 groups with 10 animals in each group. First two groups received 100 μl of Ice cold matrigel medium containing ASPC-1 (1×10⁶ cells/100 μl) and other two groups received HPAC cell lines subcutaneously. Once the tumor reached 100-120 mm³, the groups 2 and 4 received ganetespib (100 mg/kg body weight) IV once a week for three weeks. None of the animals died from the treatment. Every other day, tumor was measured using vernier caliper scale for a total of five weeks, when the animals were sacrificed. Skin around the implanted matrigel tumor was removed carefully and the tumor with its surrounding was photographed under visible light.

### CONCLUSIONS

Growth inhibition and anti-angiogenic effects of ganetespib was observed in pancreatic cancer cell lines, and ganetespib decreased HIF-1α a and VEGF expression which resulted in decrease in VEGF secretion and inhibition of angiogenesis in vivo.

### INCORPORATION BY REFERENCE

All publications, patents, and patent applications mentioned in this specification are herein incorporated by reference to the same extent as if each independent publication or patent application was specifically and individually indicated to be incorporated by reference. Moreover, this application is related to PCT application Nos. PCT/US2011/061440 and PCT/US2011/061446, both filed on Nov. 18, 2011; and to PCT application No. PCT/US12/37564, filed on May 11, 2012. Each of the applications is incorporated herein by reference.

### EQUIVALENTS

Those skilled in the art will recognize, or be able to ascertain using no more that routine experimentation, many equivalents to the specific embodiments of the invention described herein. Such equivalents are intended to be encompassed by the following claims.

1. A method for identifying a subject for treatment with an Hsp90 inhibitor comprising:
providing a subject sample from the subject,
determining the level of hypoxia in a cancer from the subject in vitro, wherein a high level of hypoxia in the sample indicates the subject is likely to respond to therapy with an Hsp90 inhibitor.

2. The method of claim 1, wherein the subject having a low level of hypoxia in the cancer is not likely to respond to therapy with an Hsp90 inhibitor.

3. The method of claim 1, wherein the cancer is a solid tumor.

4. The method of claim 1, wherein the cancer is selected from the group consisting of primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, and refractory malignancy.

5. The method of claim 1, wherein the subject sample is selected from the group consisting of tumor tissue, blood, serum, plasma, urine, stool, lymph, cerebrospinal fluid, circulating tumor cells, bronchial lavage, peritoneal lavage, exudate, effusion, and sputum.

6. The method of claim 1, wherein the level of hypoxia is determined by detecting an activity level or an expression level of one or more hypoxia-modulated peptides.

7. The method of claim 6, wherein the activity level or expression level of the one or more hypoxia-modulated polypeptides are up regulated in the sample.

8. The method of claim 1, wherein the level of hypoxia is determined by detecting the activity level or expression level of one or more hypoxia-modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

9. The method of claim 8, wherein the isoform or subunit of LDH comprises one or more selected from the group consisting of, LDH5, LDH4, LDH3, LDH2, LDH1, LDHA and LDHB; or any combination thereof including total LDH.

10. The method of claim 8, wherein the isoform of HIF is selected from the group consisting of HIF-1α, HIF-1β, HIF-2α, and HIF-2β; or any combination thereof including total HIF-1 and HIF-2.

11. The method of claim 8, wherein the pro-angiogenic isoform of VEGF is any isoform of VEGF-A; or any combination thereof including total VEGF-A.

12. The method of claim 8, wherein detection of a high level of activity or expression of at least one LDH isoform or subunit comprises detection of an LDH activity or expression level of an LDH selected from the group consisting of total LDH, LDH5, LDH4; LDH5 plus LDH4; LDH5 plus LDH4 plus LDH3; and LDHA, wherein the activity level or expression level is 0.8 ULN or more.

13. The method of claim 8, wherein detection of a high level of activity or expression of at least one LDH isoform or subunit comprises detection of an LDH activity or expression level of an LDH selected from the group consisting of total LDH, LDH5, LDH4; LDH5 plus LDH4; LDH5 plus LDH4 plus LDH3; and LDHA, wherein the activity level or expression level is 1.0 ULN or more.

14. The method of claim 1, wherein a high level of hypoxia is a change in a ratio or a ratio of normalized activity or expression levels of hypoxia-modulated polypeptides.

15. The method of claim 14, wherein a high level of hypoxia comprises a ratio or a normalized ratio of 1.0 or more of the ULN, wherein the ratio or normalized ratio is selected from the group consisting of LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

16. The method of claim 1, wherein the subject with the high level of hypoxia is administered an Hsp90 inhibitor selected from the group consisting of ganetespib, geldanamycin (tanespimycin), IPI-493, macbecins, tripterins, tanespimycins, 17-AAG (alvespimycin), KF-SS823, radicicols, KF-S8333, KF-S8332, 17-DMAG, IPI-S04, BIIB-021, BIIB-028, PU-H64, PU-H71, PU-DZ8, PU-HZ1S1, SNX-2112, SNX-2321, SNX-S422, SNX-7081, SNX-8891, SNX-0723, SAR-S67S30, ABI-287, ABI-328, AT-13387, NSC-113497, PF-3823863, PF-4470296, EC-102, EC-154, ARQ-250-RP, BC-274, VER-50589, KW-2478, BHr-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, and celastrol.

17. The method of claim 1, wherein the Hsp90 inhibitor is not ganetespib.

18. The method of claim 1, wherein the subject was previously treated with another chemotherapeutic agent.

19. Use of a level of hypoxia in a tumor for identifying a subject for treatment with an Hsp90 inhibitor comprising:
determining the level of hypoxia in a tumor from the subject, wherein a high level of hypoxia in the sample indicates the subject is likely to respond to therapy with an Hsp90 inhibitor.

20.-39. (canceled)

40. A kit for the practice of the method of claim 1.

41. A kit comprising an Hsp90 inhibitor and instruction for administration of an Hsp90 inhibitor to a subject having a tumor with a high level of hypoxia.

42. (canceled)
